(19) United States
(12) Patent Application Publication
(10) Pub. No.: US 2024/0345093 A1
(43) Pub. Date: Oct. 17, 2024

(54) METHODS AND MATERIALS FOR ASSESSING AND TREATING CANCER

(71) Applicant: The Johns Hopkins University, Baltimore, MD (US)

(72) Inventors: Bert Vogelstein, Baltimore, MD (US); Kenneth W. Kinzler, Frankford, DE (US); Qing Wang, Owings Mills, MD (US); Nickolas Papadopoulos, Towson, MD (US); Ming Zhang, Owings Mills, MD (US)

(21) Appl. No.: 18/506,393

(22) Filed: Nov. 10, 2023

Related U.S. Application Data
(63) Continuation of application No. 18/297,204, filed on Apr. 7, 2023, now abandoned.
Application No. 18/297,204 is a division of application No. 16/765,343, filed on May 19, 2020, now abandoned.
(60) Provisional application No. 62/588,654, filed on Nov. 20, 2017.

(51) Int. Cl. G01N 33/574

(52) U.S. Cl. CPC G01N 33/57449; G01N 2333/99

(57) ABSTRACT

This document provides methods and materials for identifying biomarkers (e.g., peptide biomarkers) that can be used to identify a mammal as having a disease (e.g., cancer). This document also provides methods and materials for identifying and/or treating cancer. For example, this document provides methods and materials for using one or more peptide fragments derived from a peptidyl-prolyl cis-trans isomerase A (PPIA) polypeptide to identify a mammal as having cancer (e.g., ovarian cancer).

### CROSS-REFERENCE TO RELATED APPLICATIONS

This application is a continuation application of U.S. patent application Ser. No. 18/297,204, filed on Apr. 7, 2023, which is a divisional application of U.S. patent application Ser. No. 16/765,343, filed on May 19, 2020, which is a National Stage application under 35 U.S.C. § 371 of International Application No. PCT/US2018/062007 having an International Filing Date of Nov. 20, 2018, which claims the benefit of U.S. Patent Application Ser. No. 62/588,654, filed on Nov. 20, 2017. The disclosure of the prior application is considered part of (and is incorporated by reference in) the disclosure of this application.

### SEQUENCE LISTING

This application contains a Sequence Listing that has been submitted electronically as an XML file named 44807-0302003_SL_ST26.xml. The XML file, created on Apr. 7, 2023, is 5,759,405 bytes in size. The material in the XML file is hereby incorporated by reference in its entirety.

### BACKGROUND

### 1. Technical Field

This document provides methods and materials for identifying biomarkers (e.g., peptide biomarkers) that can be used to identify a mammal as having a disease (e.g., cancer). This document also provides methods and materials for identifying and/or treating cancer. For example, this document provides methods and materials for using one or more peptide fragments derived from a peptidyl-prolyl cis-trans isomerase A (PPIA) polypeptide to identify a mammal as having cancer (e.g., ovarian cancer).

### 2. Background Information

Nearly a quarter of a million women will be diagnosed with ovarian cancer this year, and more than 140,000 women will die from their disease (Howlader et al. 2014 SEER Cancer Statistics Review, 1975-2011 (National Cancer Institute, Bethesda)). If ovarian cancer is diagnosed and treated at early stages, before the cancer has spread outside the ovary, the 5-y relative survival rate is over 90% (Howlader et al. 2014 SEER Cancer Statistics Review, 1975-2011 (National Cancer Institute, Bethesda)). However, only 15% of all ovarian cancers are found at such early stages and the prognosis for patients whose cancers are discovered at late stages is dismal (Howlader et al. 2014 SEER Cancer Statistics Review, 1975-2011 (National Cancer Institute, Bethesda)). There is thus a widely recognized need for the development of biomarkers that could potentially detect ovarian cancers earlier. There have been numerous attempts to use conventional biomarkers, such as CA-125 or HE-4, or to use ultrasound, for such detection (Fishman et al. 2005 Am J Obstet Gynecol 192:1214-1221; Li et al. 2009 Expert Rev Mol Diagn 9:555-566; Scholler et al. 2007 Biomarkers Med 1:513-523; and Van Gorp et al. 2011 Br J Cancer 104:863-870). Although some show promise, none of them is recommended for screening by the US Preventive Services Task Force because they too frequently lead to “important harms, including major surgical interventions in women who do not have cancer” (Moyer et al. 2012 Ann Intern Med 157:900-904).

### SUMMARY

This document provides methods and materials for identifying and/or treating cancer. In some cases, this document provides materials and methods for using one or more PPIA peptide fragments to identify a mammal as having cancer (e.g., ovarian cancer). For example, an elevated level of one or more PPIA peptide fragments in a sample (e.g., a non-invasive sample such as a blood sample) can be used to identify a mammal as having ovarian cancer. For example, a mammal identified as having cancer (e.g., ovarian cancer) based, at least in part, on an elevated level of one or more circulating peptide biomarkers (e.g., one or more PPIA peptide fragments) can be treated with one or more cancer treatments.

This document also provides methods and materials for identifying and/or validating peptide biomarkers (e.g., circulating peptide biomarkers) that can be used as biomarkers to identify a mammal as having cancer. In some cases, a plurality of circulating peptide biomarkers can be identified using a combination of qualitative and quantitative mass spectrometry (MS) techniques. For example, global plasma proteomic profiling of samples from cancer patients and healthy individuals can be used to identify candidate peptide biomarkers, and each candidate peptide biomarker can be evaluated by sequential analysis of fractionated eluates by selected reaction monitoring (SAFE-SRM) to validate the candidate peptide marker(s). In some cases, one or more peptides identified herein (e.g., one or more circulating peptide biomarkers), can be used to identify a mammal having a disease (e.g., cancer) as described herein.

As demonstrated herein, SAFE-SRM can be used for the discovery and validation of circulating (e.g., in the blood) peptide biomarkers for cancer. Several hundred candidate peptide biomarkers were identified through comparison of proteolytic peptides derived from the plasma of cancer patients and proteolytic peptides derived from healthy individuals, and 2D chromatography coupled with SRM was used to validate a smaller number of candidate peptide biomarkers that might prove diagnostically useful. As demonstrated herein, this approach was applied to plasma from cancer patients, and two peptides encoded by the PPIA gene were discovered whose abundance was increased in the plasma of ovarian cancer patients but not in healthy controls. This approach can be generally applied to the discovery of proteins and peptide biomarkers characteristic of any disease and/or various disease states.

Having the ability to identify peptide biomarkers in a high-throughput, robust, and reproducible system which includes validation of candidate peptide biomarkers provides a unique and unrealized opportunity to identify and validate a large number of candidate peptide biomarkers in a quantitative and massively parallel manner. In addition, having the ability to detect circulating peptide biomarkers in a blood sample provides a unique and unrealized opportunity to identify a mammal as having a cancer at earlier stages than can be achieved using conventional methods and/or using a non-invasive sample manner.

In general, one aspect of this document features a method for treating ovarian cancer. The method includes, or consists essentially of, detecting an elevated level of one or more peptide biomarkers comprising a peptide fragment derived from a PPIA polypeptide in a blood sample obtained from a mammal, and administering one or more cancer treatments to said mammal. The one or more cancer treatments can include surgery, chemotherapy, hormone therapy, targeted therapy, radiation therapy, or any combinations thereof. The mammal can be a human. The blood sample can be a plasma sample. The PPIA peptide fragment can include the amino acid sequence VSFELFADK (SEQ ID NO: 1). The PPIA peptide fragment can include the amino acid sequence FEDENFILK (SEQ ID NO: 2).

In another aspect, this document features a method for identifying a mammal as having ovarian cancer. The method includes, or consists essentially of, detecting a level of one or more blood peptide-biomarkers comprising a peptide fragment derived from a PPIA polypeptide in a blood sample obtained from said mammal, and diagnosing said mammal with ovarian cancer when an elevated level of the one or more blood peptide-biomarkers is detected in said blood sample. The mammal can be a human. The blood sample can be a plasma sample. The PPIA peptide fragment can include the amino acid sequence VSFELFADK (SEQ ID NO: 1). The PPIA peptide fragment can include the amino acid sequence FEDENFILK (SEQ ID NO: 2).

In another aspect, this document features a method for identifying a peptide biomarker. The method includes, or consists essentially of, digesting polypeptides present in a disease blood sample to obtain disease peptide fragments and labeling the disease peptide fragments with a first heavy isotope to obtain labeled disease peptide fragments; digesting polypeptides present in a reference blood sample to obtain reference peptide fragments and labeling the reference peptide fragments with a second heavy isotope to obtain labeled reference peptide fragments; and subjecting the labeled disease peptide fragments and the labeled reference peptide fragments to mass spectrometry to identify a peptide biomarker, where the level of the peptide biomarker is elevated in the labeled disease peptide fragments relative to the labeled reference peptide fragments. The disease blood sample can include blood from one or more mammals having the disease. The disease blood samples can include blood from a plurality of mammals having the disease. The reference blood sample can include blood from one or more healthy mammals. The reference blood sample can include blood from a plurality of healthy mammals. The method also can include depleting one or more highly abundant blood proteins from each sample. The highly abundant blood proteins can be albumin, IgG, α1-antitrypsin, IgA, IgM, transferrin, haptoglobin, α2-macroglobulin, fibrinogen, complement C3, α1-acid glycoprotein, apolipoprotein A-I, apolipoprotein A-II, apolipoprotein B, or any combinations thereof. The method also can include, prior to each digestion step, enriching glycoproteins in each sample. The mass spectrometry can be performed using an Orbitrap mass spectrometer.

In another aspect, this document features a method for validating a peptide biomarker. The method includes, or consists essentially of, subjecting a plurality of peptides, including the peptide biomarker, to basic pH reversed-phase liquid chromatography (bRPLC) to obtain a plurality of fractions; organizing the plurality of fractions into a plurality of fraction groups, where the number of fractions is higher than the number of fraction groups; separating peptide biomarkers in each fraction group by orthogonal high performance liquid chromatography (HPLC) at acidic pH to obtain continuous HPLC elutes; and analyzing said continuous HPLC elutes using a selected reaction monitoring (SRM) method including preoptimized transitions and preoptimized dwell times for the peptide biomarker to determine the intensity of the peptide biomarker; where the peptide biomarker is validated when the peptide biomarker is detected and quantitated at an elevated level in a disease sample relative to a reference sample using the SRM method. The method optimized dwell time for the peptide biomarker can be inversely proportional to the intensity of the peptide biomarker. The HPLC can be performed with a device that is coupled to a mass spectrometer. The mass spectrometer can be a triple quadrupole mass spectrometer. The collision energy can be any one of the collision energies set forth in Dataset S5. The dwell time can be any one of the dwell times set forth in Dataset S5.

In another aspect, this document features a method for identifying and validating a peptide biomarker. The method includes, or consists essentially of, identifying a candidate peptide biomarker, building a SAFE-SRM method for the candidate peptide biomarker, and using the SAFE-SRM method to validating the candidate peptide biomarker.

Identifying a candidate peptide biomarker can include, or consists essentially of, digesting polypeptides present in a disease blood sample to obtain disease peptide fragments, labeling the disease peptide fragments with a first heavy isotope to obtain labeled disease peptide fragments, digesting polypeptides present in a reference blood sample to obtain reference peptide fragments, labeling the reference peptide fragments with a second heavy isotope to obtain labeled reference peptide fragments, and subjecting the labeled disease peptide fragments and the labeled reference peptide fragments to mass spectrometry to identify a candidate peptide biomarker, where the level of the candidate peptide biomarker is elevated in the labeled disease peptide fragments relative to the labeled reference peptide fragments. Building a SAFE-SRM method can include, or consists essentially of,
synthesizing the candidate peptide biomarker, subjecting the synthetic candidate peptide biomarker to mass spectrometry to determine a candidate peptide biomarker transition, where the transition is determined by identifying a precursor-product ion pair having a strongest intensity and identifying a collision energy (CE) producing the precursor-product ion pair, subjecting a plurality of peptides including the candidate peptide biomarker to bRPLC to obtain a plurality of fractions, where the plurality consists of essentially equal amounts of each peptide, organizing the plurality of fractions into a plurality of fraction groups, where the number of fractions is higher than the number of fraction groups, determining an intensity of said candidate peptide biomarker in each of the fraction groups using the candidate peptide biomarker transition and a fixed dwell time, and optimizing the dwell time by re-assembling the transitions according to their hydrophobicity at high pH. Validating said candidate peptide biomarker can include, or consists essentially of, quantitating the candidate peptide biomarker in the disease blood sample, by subjecting said disease peptide fragments comprising said candidate peptide biomarkers to bRPLC to obtain a plurality of fractions, organizing said plurality of fractions into a plurality of fraction groups, where the number of fractions is higher than the number of fraction groups, separating peptides in each fraction group by orthogonal HPLC at acidic pH to obtain continuous HPLC elutes, and analyzing the continuous HPLC elutes using a SRM method including the candidate peptide biomarker transition and the optimized dwell time; and quantitating the candidate peptide marker in the reference blood sample by subjecting the reference peptide fragments to bRPLC to obtain a plurality of fractions, organizing the plurality of fractions into a plurality of fraction groups, where the number of fractions is higher than the number of fraction groups, separating peptides in each fraction group by orthogonal HPLC at acidic pH to obtain continuous HPLC elutes, and analyzing said continuous HPLC elutes using the SRM method including the candidate peptide biomarker transition and the optimized dwell time; and validating the candidate peptide biomarker when the candidate peptide biomarker is quantitated at an elevated level in the disease sample relative to the reference sample. The synthesized candidate peptide biomarkers can be not labeled with a heavy isotope. The optimized dwell time for the peptide biomarker is determined using synthetic biomarker peptides spiked and present in a sample obtained from a subject. The method optimized dwell time for the peptide biomarker can be inversely proportional to the intensity of the peptide biomarker. The HPLC can be performed with a device that is coupled to a mass spectrometer. The mass spectrometer can be a triple quadrupole mass spectrometer. The collision energy can be any one of the collision energies set forth in Dataset S5. The dwell time can be any one of the dwell times set forth in Dataset S5.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains schematics of workflow of plasma biomarker identification and validation. Plasma biomarker discovery and identification were conducted through labeling-dependent quantitative proteomics, such as iTRAQ or TMT assays (A); plasma biomarker validation was conducted through SAFE-SRM (B).

FIG. 2 shows peptide detectability by SAFE-SRM in complex samples. Six heavy-isotope-labeled peptides (peptide 1: IQLVEEELDR* (SEQ ID NO:3); peptide 2: VILHLK* (SEQ ID NO:4); peptide 3: IILLFDAHK* (SEQ ID NO:5); peptide 4: TLAESALQLLYTAK* (SEQ ID NO:6); peptide 5: LLGHLVK* (SEQ ID NO:7); peptide 6: GLVGEIIK* (SEQ ID NO:8), where * indicates C13 and N15 heavy-isotope-labeled amino acids) were synthesized and used to evaluate the sensitivity of SAFESRM in detecting low amount of peptides in complex samples. One femtomole of each peptide was detected by conventional SRM (A). However, when 1 fmol of these peptides was added to trypsin-digested plasma samples, they were much more difficult to detect (B). bRPLC fractionation was able to increase the sensitivity of standard SRM, but with a large variance between runs (C). SAFE-SRM with optimized dwell and cycling time allowed detection of all six peptides, at intensities averaging 70% of the intensities of the free peptides (D).

FIG. 3 shows ovarian cancer prediction by peptide biomarkers. (A) Mean square errors (MSEs) of ovarian cancer prediction of all 318 peptides are plotted with the peptides ranked by MSE from the best predictors to the worst predictors. (B) The 10 best peptide biomarkers are shown; the peptide VSFELFADK from peptidyl-prolyl cis-trans isomerase A was the best predictor. (C) The ovarian cancer prediction performance of PPIA peptide VSFELFADK was further improved by combining with another peptide, FEDENFILK (SEQ ID NO: 2), from the same protein.

FIG. 4 contains a detailed technical workflow for iTRAQ-labeling-based quantitative proteomics studies with total plasma proteome (A) and plasma glycoproteome (B).

FIGS. 5A-5C contain a SAFE-SRM scheme. (A) bRPLC fractionation was performed to separate peptides from a complicated biological sample into 96 fractions according to their hydrophobicity at high pH. The SAFE-SRM fraction groups are overlaid on the wells. (B) A chromatogram showing the combined signal intensities of all peptides in each of the 20 SAFE-SRM fraction groups used in the final SAFE-SRM method. (C) SAFE-SRM method transition coverages. For each fraction group i, the specific SAFE-SRM method i is composed of the transitions detecting peptides within that fraction group and two adjacent groups, group i−1 and group i+1, where i∈.

FIG. 6 contains SAFE-SRM profiles for three ovarian cancer biomarker peptides in eight plasma samples. Four ovarian cancer plasma samples (253, 256, 260, and 271) and four normal healthy plasma samples (202, 205, 207, and 209) were analyzed by SAFE-SRM. The areas under the peak are shown for each sample.

FIG. 7 contains a comparison of ovarian cancer diagnostic performance using SAFE-SRM-based PPIA assay and ELISA-based CA125 assay. The Venn diagram shows the number of cases identified in a cohort of 63 ovarian cancer patients.

FIG. 8 contains MS spectra of SAFE-SRM target peptides from PPIA.

FIG. 9 contains MA plots for whole-plasma iTRAQ datasets. Nonnormalized peptide intensities from each of the three experiments were compared under each specific labeling (114, 115, 116, and 117) and corresponding MA plots were generated using the log-transformed raw intensities, with A ranges fixed to 6-14, and M ranges fixed to −4 to 4. There is no clear evidence of bias associated with any of the datasets. The technical variance (I-L) is significantly smaller than the biological variance (A-D or E-H).

FIG. 10 shows nonnormalized and median normalized histograms for cancer vs. normal in three datasets. Protein ratios of cancers/normal were plotted using log 2 scale for dataset 1 (A-C, Upper), dataset 2 (A-C, Middle), and dataset 3 (A-C, Lower). After median normalization, the same protein ratios of cancers/normal were plotted using log 2 scale for dataset 1 (D-F, Upper), dataset 2 (D-F, Middle), and dataset 3 (D-F, Lower). The log 2 (relative ratio)=0 lines are indicated in each plot (red line). Biased data were observed for colorectal cancer (B) and ovarian cancer (C). The bias for pancreatic cancer (A) is not obvious.

### DETAILED DESCRIPTION

This document provides methods and materials for identifying and/or treating a disease. In some cases, the disease is cancer. For example, a mammal having an elevated level of one or more circulating peptide biomarkers (e.g., PPIA peptide fragments) can be identified as having cancer (e.g., ovarian cancer) and, optionally, can be administered one or more cancer treatments. As used herein a “circulating peptide” is a peptide that can be detected in any closed system (e.g., the circulatory system) within the body of a mammal. In some cases, a blood sample (e.g., a plasma sample) from a mammal (e.g., a mammal suspected as having cancer) can be assessed for an elevated level of one or more PPIA peptide fragments, and when an elevated level of one or more PPIA peptide fragments is detected, the mammal can be identified as having cancer, and, optionally, the mammal can be administered one or more cancer treatments to reduce the severity of the cancer and/or to reduce a symptom of the cancer.

The term “elevated level” as used herein with respect to a level of a circulating peptide biomarker (e.g., a PPIA peptide fragment) refers to any level that is greater than the reference level of the circulating peptide (e.g., PPIA peptide fragment) typically observed in a sample (e.g., a reference sample) from one or more healthy mammals (e.g., mammals that do not have a cancer). In some cases, a reference sample can be a sample obtained from a mammal that does not exhibit the disease that is associated with an elevated level of a circulating peptide. For example, for a peptide biomarker associated with ovarian cancer, a reference sample can be a sample obtained from a subject that does not have ovarian cancer. In some cases, a reference sample can be a sample obtained from the same mammal in which the elevated level of a peptide biomarker is observed, where the reference sample was obtained prior to onset of the disease that is associated with an elevated level of a circulating peptide. In some cases, such a reference sample obtained from the same mammal is frozen or otherwise preserved for future use as a reference sample. In some cases, an elevated level of one or more PPIA fragments can be assessed based on an abundance score thresholds as described herein (see, e.g., Examples 1 and Dataset S7). In some cases, when reference samples have undetectable levels of a circulating peptide biomarker, an elevated level can be any detectable level of the circulating peptide biomarker. It will be appreciated that levels from comparable samples are used when determining whether or not a particular level is an elevated level.

Any appropriate mammal can be assessed and/or treated as described herein. For example, humans or other primates such as monkeys can be assessed for an elevated level of one or more PPIA peptide fragments and, optionally, can be treated with one or more cancer treatments to reduce the number of cancer cells present within the human or other primate. In some cases, dogs, cats, horses, cows, pigs, sheep, mice, and rats having cancer can be assessed for an elevated level of one or more PPIA peptide fragments, and, optionally, can be treated with one or more cancer treatments to reduce the number of cancer cells present within the human or other primate as described herein.

Any appropriate sample from a mammal can be assessed as described herein (e.g., assessed for an elevated level of one or more circulating peptide biomarkers). Examples of samples that can contain circulating peptide biomarkers include, without limitation, blood samples (e.g., whole blood, serum, or plasma samples), blood, plasma, urine, cerebrospinal fluid, saliva, sputum, broncho-alveolar lavage, bile, lymphatic fluid, cyst fluid, stool, and ascites. In some cases, a sample can be a plasma sample.

The one or more circulating peptide biomarkers can be any appropriate circulating peptide biomarker. In some cases, circulating peptide biomarkers are identified and validated using any of the methods described herein (e.g., using a SAFE-ARM method). The one or more PPIA peptide fragments can include any appropriate PPIA peptide fragments. Examples of PPIA peptide fragments include, without limitation, peptide fragments that include the amino acid sequence VSFELFADK (SEQ ID NO: 1) and peptide fragments that include the amino acid sequence FEDENFILK (SEQ ID NO: 2).

Any appropriate method can be used to detect an elevated level of one or more circulating peptide biomarkers. Examples of methods for detecting peptide levels include, without limitation, spectrometry methods (e.g., high-performance liquid chromatography (HPLC) and liquid chromatography-mass spectrometry (LC/MS)), antibody dependent methods (e.g., enzyme-linked immunosorbent assay (ELISA), protein immunoprecipitation, immunoelectrophoresis, western blotting, and protein immunostaining), and aptamer dependent methods. In some cases, one or more circulating peptide biomarkers (e.g., one or more PPIA peptide fragments) can be detecting using mass spectrometry techniques.

In some cases, a mammal identified as having cancer as described herein (e.g., based at least in part on an elevated level of one or more circulating peptide biomarkers) can have the cancer diagnosis confirmed using any appropriate method. Examples of methods that can be used to diagnose a cancer include, without limitation, physical examinations (e.g., pelvic examination), imaging tests (e.g., ultrasound or CT scans), blood tests (e.g., for markers such as CA 125), tissue tests (e.g., biopsy).

Once identified as having a cancer as described herein (e.g., based at least in part on an elevated level of one or more circulating peptide biomarkers such as PPIA peptide fragments), a mammal can be treated with one or more cancer treatments. The one or more cancer treatments can include any appropriate cancer treatments. A cancer treatment can include surgery. In cases where the cancer is ovarian cancer, surgery can include removal of one or both ovaries, the fallopian tubes, the uterus, nearby lymph nodes, and/or nearby fatty abdominal tissue (omentum). A cancer treatment can include radiation therapy. A cancer treatment can include administration of a pharmacotherapy such chemotherapy, hormone therapy, targeted therapy, and/or cytotoxic therapy. Examples of cancer treatments include, without limitation, platinum compounds (such as cisplatin or carboplatin), taxanes (such as paclitaxel or docetaxel), albumin bound paclitaxel (nab-paclitaxel), altretamine, capecitabine, cyclophosphamide, etoposide (vp-16), gemcitabine, ifosfamide, irinotecan (cpt-11), liposomal doxorubicin, melphalan, pemetrexed, topotecan, vinorelbine, luteinizing-hormone-releasing hormone (LHRH) agonists (such as goserelin and leuprolide), anti-estrogen therapy (such as tamoxifen), aromatase inhibitors (such as letrozole, anastrozole, and exemestane), angiogenesis inhibitors (such as bevacizumab), poly (ADP)-ribose polymerase (PARP) inhibitors (such as olaparib, rucaparib, and niraparib), external beam radiation therapy, brachytherapy, radioactive phosphorus, and any combinations thereof.

Any appropriate cancer can be identified and/or treated as described herein. Examples of cancers that can be treated as described herein include, without limitation, lung cancer (e.g., small cell lung carcinoma or non-small cell lung carcinoma), papillary thyroid cancer, medullary thyroid cancer, differentiated thyroid cancer, recurrent thyroid cancer, refractory differentiated thyroid cancer, lung adenocarcinoma, bronchioles lung cell carcinoma, multiple endocrine neoplasia type 2A or 2B (MEN2A or MEN2B, respectively), pheochromocytoma, parathyroid hyperplasia, breast cancer, colorectal cancer (e.g., metastatic colorectal cancer), papillary renal cell carcinoma, ganglioneuromatosis of the gastroenteric mucosa, inflammatory myofibroblastic tumor, or cervical cancer, acute lymphoblastic leukemia (ALL), acute myeloid leukemia (AML), cancer in adolescents, adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, colon cancer, colorectal cancer, craniopharyngioma, cutaneous T-cell lymphoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, myelogenous leukemia, myeloid leukemia, multiple myeloma, myeloproliferative neoplasms, nasal cavity and paranasal sinus cancer, nasopharyngeal cancer, neuroblastoma, non-Hodgkin's lymphoma, non-small cell lung cancer, oral cancer, oral cavity cancer, lip cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromosytoma, pituitary cancer, plasma cell neoplasm, pleuropulmonary blastoma, pregnancy and breast cancer, primary central nervous system lymphoma, primary peritoneal cancer, prostate cancer, rectal cancer, renal cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, Waldenstrom Macroglobulinemia, and Wilms' tumor. In some cases, the materials and methods described herein can be used to identify and/or treat ovarian cancer.

In another aspect, this document also provides methods and materials for identifying and/or validating peptide biomarkers (e.g., circulating peptide biomarkers) that can be used to identify a mammal as having a disease and/or disease stage. In some cases, methods and materials provided herein can be used for identifying and/or validating peptide biomarkers (e.g., circulating peptide biomarkers) that can be used to identify a mammal as having cancer.

Methods and materials described herein can be used for identifying a peptide biomarker (e.g., a circulating peptide biomarker). In some cases, methods for identifying circulating peptide biomarkers can include identifying circulating peptide biomarkers that are elevated in a disease sample as compared to a control sample (e.g., a reference sample). In some cases, a disease sample can include blood from one or more (e.g., 2, 3, 5, 8, 10, 11, 12, 13, 14, 15, 16, 17, 18, 19, 20, 25, 30, 35, 40, 50, 60, 70, 80, 90, 100 or more) mammals having a disease. In some cases, a disease sample can include blood from a single mammal. In some cases, a control sample can include blood from one or more (e.g., 2, 3, 5, 8, 10, 11, 12, 13, 14, 15, 16, 17, 18, 19, 20, 25, 30, 35, 40, 50, 60, 70, 80, 90, 100 or more) healthy mammals (e.g., mammals that do not have a disease). In some cases, a control sample can include blood from a single mammal. In some cases, a method for identifying one or more circulating peptide biomarkers can include digesting polypeptides present in a disease blood sample into peptide fragments to obtain a sample of disease peptide fragments; and digesting polypeptides present in a reference blood sample into peptide fragments to obtain a sample of reference peptide fragments. In some cases, the peptide fragments from a digested sample (e.g., the disease peptide fragments or the reference peptide fragments) can be differentially labeled. For example, the peptide fragments from a disease blood sample can remain label-free and the peptide fragments from a reference sample can be labeled with a heavy isotope, or vice versa. For example, the peptide fragments from a disease blood sample and the peptide fragments from a reference sample can be labeled with different heavy isotopes. In some cases, one or more (e.g., 2, 3, 4, 5, or more) samples from different diseases (e.g., different cancer types) or different disease stages (e.g., a first disease sample being an early disease sample and a second disease sample being an advanced disease sample) can be used, and each sample (e.g., each disease sample and the control sample) can each be labelled with a difference heavy isotope. Examples of heavy isotopes include, without limitation, deuterium, C13, N15, and O18. When the peptide fragments from a disease blood sample and the peptide fragments from a reference sample are not labeled, the disease peptide fragments and the reference peptide fragments can be subjected to mass spectrometry (e.g., independently subjected to mass spectrometry as separate runs), and the results can be compared to identify one or more peptide biomarkers (e.g., peptides that are elevated in the disease sample relative to the reference sample). When the peptide fragments from a disease blood sample and the peptide fragments from a reference sample are differentially labeled, the labeled disease peptide fragments and the labeled reference peptide fragments can be subjected to mass spectrometry (e.g., as a single mass spectrometry run) to identify one or more peptide biomarkers (e.g., peptides that are elevated in the disease sample relative to the reference sample).

Any appropriate mass spectrometer can be used. Examples of mass spectrometers include, without limitation, an Orbitrap mass spectrometer and a triple quadrupole mass spectrometer, time-of-flight (TOF), matrix-assisted laser desorption/ionization (MALDI)-TOF, and surface-enhanced laser desorption/ionization (SELDI)-TOF. For example, an Orbitrap mass spectrometer can be used when identifying one or more peptide biomarkers as described herein.

Any appropriate method for digesting polypeptides can be used. In some cases, polypeptides can be enzymatically digested. In some cases, polypeptides can be chemically digested. For example, polypeptides can be digested using, without limitation, Arg-C, Asp-N, Asp-N(N-terminal Glu), BNPS or NCS/urea, Caspase-1, Caspase-10, Caspase-2, Caspase-3, Caspase-4, Caspase-5, Caspase-6, Caspase-7, Caspase-8, Caspase-9, Chymotrypsin, Chymotrypsin (low specificity), Clostripain, CNBr, CNBr (methyl-Cys), CNBr (with acids), Enterokinase, Factor Xa, Formic acid, Glu-C (AmAc buffer), Glu-C (Phos buffer), Granzyme B, HRV3C protease, Hydroxylamine, Iodosobenzoic acid, Lys-C, Lys-N, Lys-N(Cys modified), Mild acid hydrolysis, NBS (long exposure), NBS (short exposure), NTCB, Pancreatic elastase, Pepsin A, Pepsin A (low specificity), Prolyl endopeptidase, Proteinase K, TEV protease, Thermolysin, Thrombin, Trypsin and/or hydrolysis.

In some cases, methods for identifying one or more circulating peptide biomarkers can include reducing or eliminating circulating proteins that are present in high abundance from the disease sample and/or the control sample. Examples of circulating proteins that are present in high abundance include, without limitation, albumin, immunoglobulins (e.g., IgG, IgA, and IgM), α1-antitrypsin, transferrin, haptoglobin, α2-macroglobulin, fibrinogen, complement C3, α1-acid glycoprotein (Orosomucoid), high-density lipoproteins (HDLs; e.g., apolipoproteins A-I and A-II), and low-density lipoproteins (LDLs; e.g., apolipoprotein B). Circulating proteins that are present in high abundance can be reduced or eliminated using any appropriate technique. Circulating proteins can be reduced or eliminated using any appropriate technique. Examples of means for reducing or eliminating circulating proteins include, without limitation, cibacron blue dye and antibody-based plasma depletion. For example, circulating proteins that are present in high abundance can be reduced or eliminated by antibody-based plasma depletion.

In some cases, methods for identifying one or more circulating peptide biomarkers can include enriching circulating proteins that are present in low abundance from the disease sample and/or the control sample. For example, low abundance proteins can be enriched using a peptide ligand library (see, e.g., the strategy in ProteoMiner protein enrichment kit) or using aptamers.

In some cases, methods for identifying one or more circulating peptide biomarkers can include denaturing, reducing, and/or alkylating the peptide fragments from a disease blood sample and/or a control sample. For example, peptides can be denatured using urea, sodium dodecyl sulfate (SDS), methanol, glycerol, and/or heat. For example, peptides can be reduced using tris-(2-carboxyethyl) phosphine (TCEP), dithiothreitol (DTT), and/or 2-mercaptoethanol. For example, peptides can be alkylated using methyl methanethiosulfonate (MMTS), iodoacetamide, and/or iodoacetate.

In some cases, methods for identifying one or more circulating peptide biomarkers can include enriching glycoproteins, phosphorylated proteins, and/or proteins bearing other post-translation modifications in each sample.

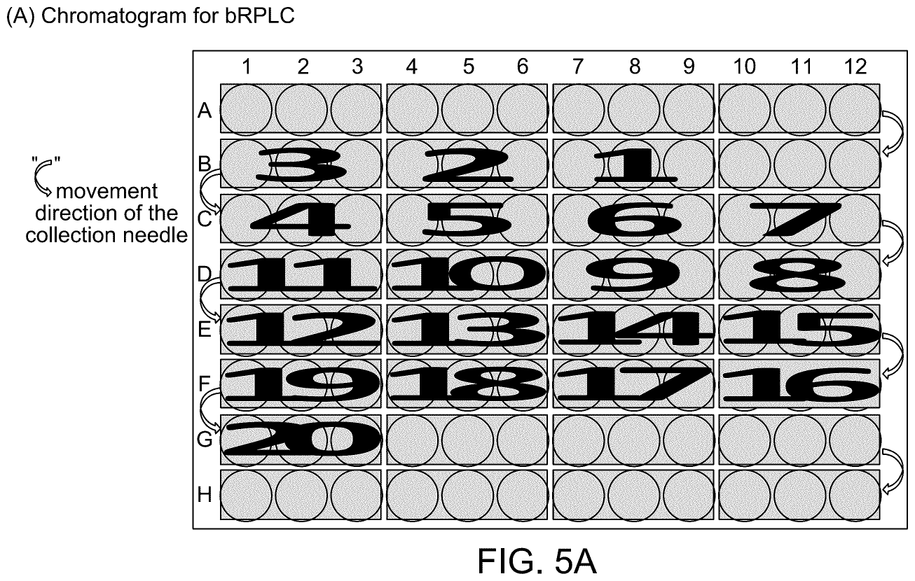

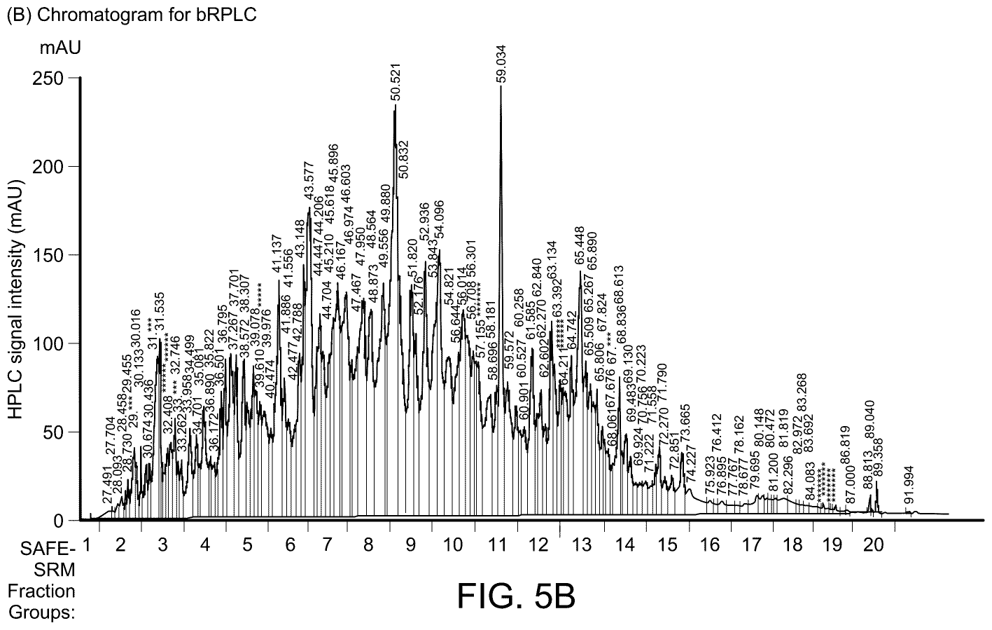

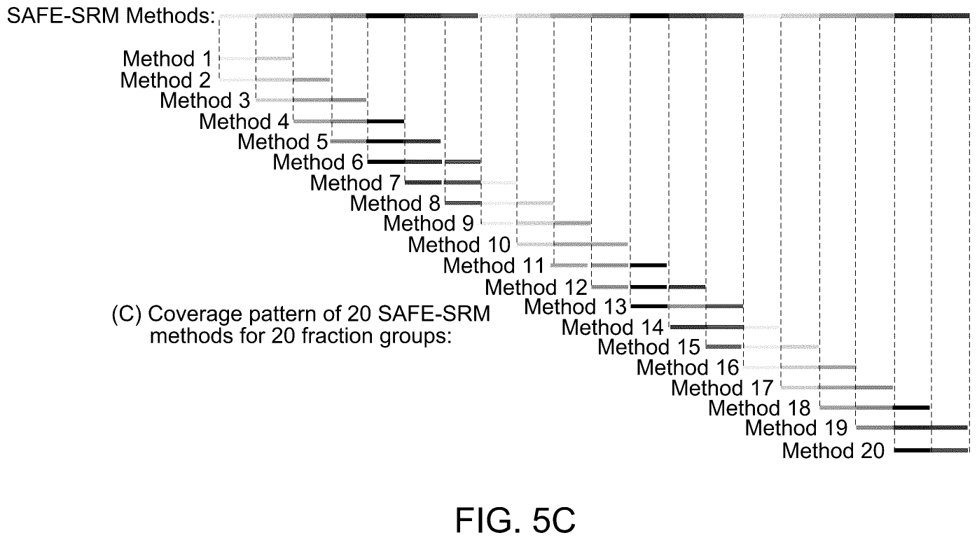

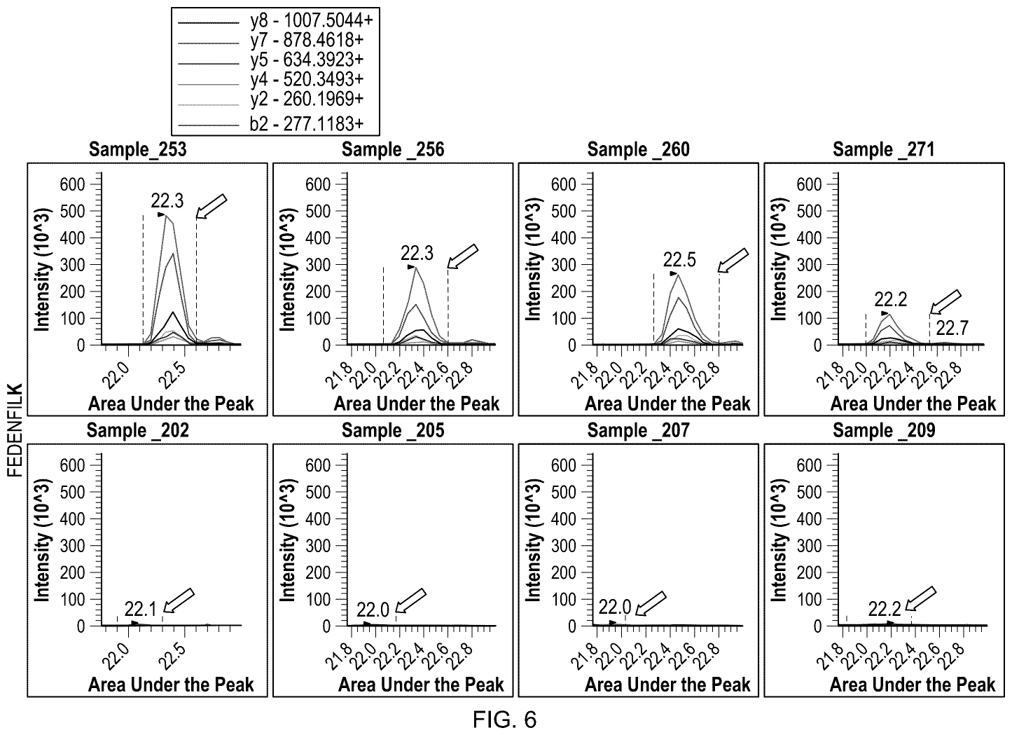

Methods and materials described herein can be used for validating a peptide biomarker (e.g., a circulating peptide biomarker). In some cases, methods for validating one or more circulating peptide biomarkers can include validating circulating peptide biomarkers that have been identified according to any of the variety of methods described herein. Methods for validating a peptide biomarker (e.g., a circulating peptide biomarker) can include a sequential analysis of fractionated eluates by selected reaction monitoring SRM (SAFE-SRM). In some cases, a peptide biomarker can be validated using a SRM method including preoptimized transitions and/or preoptimized dwell times (e.g., to determine the intensity of the peptide biomarker). In some cases, a peptide biomarker can be validated by building a SRM method having optimized transitions and/or optimized dwell times for determining the intensity of the peptide biomarker. For example, for each set of candidate peptide biomarkers, a set of SAFE-SRM methods can be compiled. As demonstrated in Example 1, synthetic peptides of each candidate biomarker can be subjected to basic pH reversed-phase liquid chromatography (bRPLC) and generate fraction groups. The fraction groups of synthetic peptides can be subjected to mass spectrometry to determine which synthetic peptides are located in which groups, and at the same time determine, within its group, the standard intensity of the peptide (as derived from the certain amount initially used) (see, e.g., FIG. 5). A peptide biomarker can be validated, for example, when the peptide biomarker is detected and quantitated at an elevated level in a disease sample relative to a reference sample using a SAFE-SRM method described herein.

In some cases, methods for validating a peptide biomarker (e.g., using SAFE-SRM) can include subjecting one or more peptide biomarkers to bRPLC (e.g., bRPLC at high pH) to obtain a plurality of fractions (e.g., 5, 10, 15, 20, 25, 30, 35, 40, 45, 50, 55, 60, 65, 70, 75, 80, 85, 90, 95, 100, 110, 120, 130, 140, 150, 160, 170, 180, 190, 200 or more fractions); organizing the plurality of fractions into a plurality of fraction groups (e.g., 2, 3, 5, 10, 15, 20, 25, 30, 35, 40, 45, 50 or more fraction groups); separating the peptide biomarkers in each fraction group by orthogonal HPLC at acidic pH (low pH) to obtain continuous HPLC elutes; and analyzing the continuous HPLC elutes using a SRM method, wherein the peptide biomarker is validated when a collision energy, a dwell time optimized for the peptide biomarker is observed. In some cases, the SRM method can be pre-established with the synthetic peptides eluted in that fraction group. In some cases, the plurality of fractions includes 48, 96, or 384 fractions. In some cases, the plurality of fraction groups includes 16, 32, or 124 fraction groups.

In some cases, methods for validating a peptide biomarker (e.g., using SAFE-SRM) can include coupling the HPLC to a mass spectrometer. Any appropriate mass spectrometer can be used. Examples of mass spectrometers include, without limitation, an Orbitrap mass spectrometer, a triple quadrupole mass spectrometer, TOF, MALDI-TOF, and SELDI-TOF. For example, the HPLC can be coupled to a triple quadrupole mass spectrometer can be used when validating one or more peptide biomarkers as described herein.

In some cases, methods for validating a peptide biomarker (e.g., using SAFE-SRM) can include building transition parameters for each peptide biomarker. For example, a transition can include, without limitation, parameters of precursor ion m/Z, product ion m/Z, collision energy, and/or dwell time. A transition can be optimized for a specific precursor-product ion pair. For example, each peptide that is a precursor can have multiple product ions after being fragmented, and each product ion can have its own optimized collision energy and dwell time. In some cases, optimizing the dwell time can include re-assembling the transitions according to their hydrophobicity at high pH (see, e.g., Example 1 and FIG. 5). In some cases, when optimizing the dwell time, different target peptides can be spiked at about the same amount, to determine which peptides may need to be detected with a longer dwell time. Each peptide can have several transitions where each transition corresponds to a precursor-product ion pair. In some cases, transitions can be optimized for each target peptide using a synthetic peptide. In some cases, transition parameters can be as set forth in Dataset S5.

In some cases, fractions before and after any given fraction can be analyzed to balance out the potential fluctuation of the bRPLC retention time in analyzing numerous samples.

In some cases, methods for validating a peptide biomarker (e.g., using SAFE-SRM) can be established with synthetic peptides.

In some cases, methods for validating a peptide biomarker (e.g., using SAFE-SRM) can be established with light peptides (e.g., peptides that are not labeled with a heavy isotope). Use of light peptides can be advantageous for any of a variety of reasons. For example, light peptides are generally less costly to produce, and their use thus reduces the high cost of using heavy peptides, particularly in the early stages of biomarker development where hundreds or thousands of biomarkers need to be validated. Heavy-isotope-labeled peptides may also lead to ion suppression, thereby compromising sensitivity.

The methods and materials described herein can be used for both identifying a peptide biomarker (e.g., a circulating peptide biomarker) and validating a peptide biomarker (e.g., using SAFE-SRM).

The invention will be further described in the following examples, which do not limit the scope of the invention described in the claims.

### EXAMPLES

### Example 1: Selected Reaction Monitoring Approach for Validating Candidate Biomarkers

This example describes a peptide-centric platform for developing unique biomarkers that can narrow down a large list of candidate peptides to a more manageable list that does not compromise quantification, sensitivity, or specificity. This example further shows that peptides isolated directly from plasma, rather than from cancer tissues, can be used for the discovery of unique cancer biomarkers.

### Materials and Methods

Plasma Samples. Plasma samples from a total of 266 individuals were obtained, comprising 96 healthy individuals, 81 patients with ovarian cancer, 51 with pancreatic cancer, and 38 with colorectal cancer. The plasma samples and clinical data were obtained from The Ontario Tumor Bank, Indivumed, Innovative Research, and The Johns Hopkins Hospital after appropriate institutional review board approval. Selected clinical features of the 266 patients and histopathologic characteristics of their tumors are listed in Dataset S1.

Materials and Reagents. Human plasma depletion Seppro IgY14 LC10 column systems were purchased from Sigma-Aldrich. Tris-(2-carboxyethyl) phosphine (TCEP) and methyl methanethiosulfonate (MMTS) were purchased from Thermo Fisher Scientific. LysC and Trypsin proteases were purchased from Promega. PNGase F was purchased from New England Biolabs. Titansphere, 10 or 5 μm, for TiO2 enrichment was obtained from GL Sciences. CA19-9 and CA125 antibodies were purchased from Fujirebio Diagnostics. PolySULFOETHYL A column (100×2.1 mm, 5 μm, 200 Å) for strong cation exchange (SCX) chromatography was purchased from PolyLC. C18 Cartridges for sample preparation and chromatography columns for bRPLC and online HPLC of triple-quadrupole mass spectrometer were purchased from Waters. All iTRAQ reagents and buffers were purchased from AB Sciex. Synthetic peptides were purchased from Genscript. All other reagents were purchased from Sigma-Aldrich, unless otherwise indicated.

Preparation of Solutions. SCX solvent A contained 10 mM KH2PO4, 25% (vol/vol) acetonitrile; SCX solvent B contained 10 mM KH2PO4, 350 mM KCL, 25% (vol/vol) acetonitrile; and for both SCX solvents, pH 2.75 was achieved by adding 50% H3PO4. bRPLC solvent A contained 10 mM TEABC; bRPLC solvent B contained 10 mM TEABC, 90% (vol/vol) acetonitrile. SAFE-SRM MS solvent A was water with 0.1% (vol/vol) formic acid; SAFESRM solvent B was acetonitrile with 0.1% (vol/vol) formic acid.

Pooled Plasma Samples for iTRAQ-Based Discovery Studies. Fifty normal individuals, 13 patients with pancreatic cancer, 18 with colorectal cancer, and 18 with ovarian cancer were chosen for initial analysis. One hundred microliters of plasma from each individual in one of these four groups of patients was pooled before processing through phase 1 of the study. Phase 1 of this study used these pools rather than peptides from individual patients and are referred to as “pooled peptides.”

Plasma Depletion. Abundant proteins [albumin, IgG, al-antitrypsin, IgA, IgM, transferrin, haptoglobin, α2-macroglobulin, fibrinogen, complement C3, α1-acid glycoprotein (orosomucoid), HDL (apolipoproteins A-I and A-II), and LDL (mainly apolipoprotein B)] in the plasma were depleted using a Seppro IgY14 LC10 column system. Plasma samples were diluted 5× in IgY dilution buffer, filtered (0.22 μm), and then injected into IgY LC10 columns attached to an Agilent 1200 HPLC system consisting of a binary pump, external sample injector, UV detector, and a fraction collector. The nonretained fraction was collected.

Plasma Proteome Sample Preparation. The depleted plasma proteins were denatured in 9 M urea, reduced using 5 mM TCEP at 60° C. for 15 min, and cysteine residues were alkylated with 5 mM MMTS for 15 min at room temperature in dark. The alkylated protein solution was filtered to desalt using the Amicon Ultra-15 Centrifugal Filter Unit with Ultracel-10 membrane (Millipore) and washed with 9Murea for two times, and the desalted plasma protein was reconstituted with 4 mL of 40 mM TEABC. The samples were then digested for 3 h with LysC protease followed by an overnight digestion using sequencing-grade trypsin at 37° C. Additional sequencing-grade trypsin was added 3 h before digestion ended, and the digestion system was incubated at 50° C. for the last 30 min before adding 1% TFA to stop the reaction. C18-mediated cleaning of the digest was performed as described elsewhere (see, e.g., Howlader et al. 2014 SEER Cancer Statistics Review, 1975-2011 (National Cancer Institute, Bethesda)). For samples not used in iTRAQ experiments, that is, those from individual donors rather than pooled plasma samples, 50 mM iodoacetamide (Sigma-Aldrich) rather than MMTS was used for alkylation.

N-Glycosylated Protein Enrichment and Isolation from Human Plasma Samples. One hundred microliters of pooled human plasma samples was denatured in 9 M urea and processed through reduction, alkylation, and filtration to remove salt, and then subjected to lyophilization. Lyophilized proteins were reconstituted with 5% acetonitrile with 0.1% TFA. The 10 mM sodium periodate was applied to the protein solution followed by incubation at 4° C. for 1 h in the dark. Another C8 cartridge cleaning was performed to purify the oxidized proteins. Lyophilized proteins were reconstituted with 1 mL of hydrazide resin coupling buffer (0.1 M sodium phosphate buffer, pH 7.0), and 250 μL of hydrazide resin, purchased from Bio-Rad, was added to the solution to conjugate the glycoproteome by incubation at room temperature for 5 h. The resin was then washed twice with 4 mL of 1.5 M NaCl followed by 4 mL of water, twice with 4 mL of 100 mM TEABC buffer, and finally with 4 mL of 50 mM sodium phosphate (pH 7.5). Twenty-five microliters of PNGase F was added to the resin followed by incubation at 37° C. for 4 h with agitation. The resin was then centrifuged at 8,000×g for 5 min, and the supernatant was collected. The resin pellet was washed twice with 500 μL of 40 mM ammonium bicarbonate and subjected to centrifugation as above. The supernatants from these centrifugations were combined, lyophilized, and reconstituted with 40 mM ammonium bicarbonate, and subject to trypsin digestion and C18 cleaning, after which they were used for iTRAQ labeling. A total of 657 glycosylated proteins was identified and quantified (Dataset S3). There were 29 proteins identified from the N-glycosylated protein enrichment experiments that were carried forward to the validation phases of this study.

iTRAQ Labeling, SCX Cleaning, and bRPLC Fractionation. Peptides from the four pools were reconstituted in 15 μL of H2O and 20 μL of dissolution buffer (provided with the iTRAQ labeling kit) and incubated with one of the four iTRAQ reagents diluted in 70 μL of ethanol at room temperature. The peptides from each of the four pools were labeled with iTRAQ reagents containing 114, 115, 116, or 117 reporter ions, respectively. After incubation at room temperature for 2 h, 50 μL of water was added. After another incubation for 10 min at room temperature, 100 μL of water was added. After incubation at room temperature for another 10 min, 40 μL of 40 mM ammonium bicarbonate was then added, and the reactions were incubated at 4° C. overnight. The samples were vacuum dried to 50 μL, combined, and diluted to 4 mL in 10 mM potassium phosphate buffer (pH 2.7) containing 25% acetonitrile (SCX solvent A). The pH of the sample was adjusted to 2.7 using 100 mM phosphoric acid. iTRAQ-labeled peptides were then purified using SCX chromatography with a polysulfoethyl A column (PolyLC) (300 Å, 5 μm, 100×2.1 mm) (see, e.g., Fishman et al. 2005 Am J Obstet Gynecol 192:1214-1221) on an Agilent 1200 HPLC system. Fractionation was carried out for a period of 45 min using a linear gradient of increasing salt concentration from 0 to 350 mM KCl in SCX solvent B. Peptide fractionations were then vacuum dried and reconstituted with 4 mL of bRPLC solvent A and subject to bRPLC fractionation with an XBridge C18 column (Waters). A total of 96 fractions from the bRPLC was deposited in a 96-well plate.

Plasma Peptide Preparation. The 200-μL plasma samples from each individual were processed using the procedures described above. Lyophilized plasma peptide samples were reconstituted in 2 mL of 10 mM triethylammonium bicarbonate (pH 8.2) with 3% acetonitrile. Peptide fractionation was performed on an Agilent 1260 HPLC system with a C18 column at high pH. The two HPLC mobile phase solvents were 10 mM triethylammonium bicarbonate (solvent A), and 10 mM triethylammonium bicarbonate with 90% acetonitrile (solvent B). A 120-min HPLC gradient method was applied with a flushing step for the first 20 min to remove salt, and this was followed by a 96-min gradient with solvent B increasing from 0 to 100%. The 96 fractions from a plasma peptide sample were collected in a Protein LoBind plate (Eppendorf), and the peptides eluted during each 1-min window were collected in each well. Peptide fractions were combined according to the scheme shown in FIG. 5A and vacuum dried. Dried peptides were then reconstituted using 40 μL of SRMsolvent A and spiked with 3 fmol of heavy isotope-labeled K-Ras wild-type (WT) peptides (LVVVGAGGVGK*; SEQ ID NO:23) before another online fractionation on an Agilent 1290 UHPLC system. The online UHPLC fractionated each sample at low pH (pH 3), which created a dramatically different fractionation profile than the first HPLC fraction, which was performed at high pH (pH 8.2). Fractionated samples were continuously injected into the Jet Stream ESI source of an Agilent 6490 triple-quadrupole mass spectrometer operated in SRM positive-ion mode.

Quantitative Proteomics Assays for Normal and Cancer Plasma Samples. iTRAQ labeling-dependent quantitative proteomics assays were performed to evaluate the proteomic difference between normal plasma and cancer plasma samples. The pipeline included plasma depletion, denaturation, reduction, alkylation, enrichment for glycoproteins, trypsin digestion, desalting, iTRAQ labeling, strong cation exchange (SCX) cleaning, and bRPLC fractionation followed by Orbitrap MS analysis and quantitative proteomics data analysis using in-house-developed R scripts.

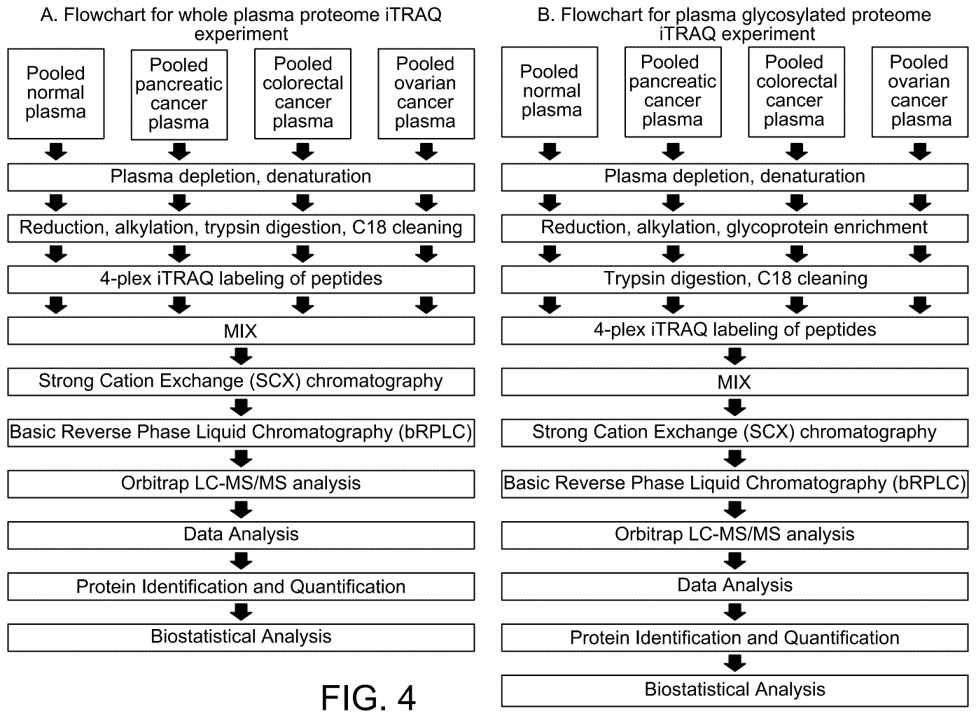

Liquid Chromatography-MS/MS and Plasma Quantitative Proteomics Data Analysis. Nanoflow electrospray ionization liquid chromatography (LC)-MS/MS analysis of the iTRAQ-labeled bRPLC-separated samples was performed with an LTQ Orbitrap Velos (Thermo Fisher Scientific) mass spectrometer interfaced with reversed-phase system controlled by Eksigent nano-LC and Agilent 1100 microwell plate autosampler. The bRPLC fractions were sequentially processed through a 75 μm×2 cm, Magic C18AQ column (5 μm, 100 Å; Michrom Bioresources) and then separated on an analytical column (75 μm×10 cm, Magic C18AQ, 5 μm, 100 Å; Michrom Bioresources) with a nanoflow solvent delivery. The mobile phase flow rate was 200 nL/min, composed of 3% acetonitrile/0.1% formic acid (solvent A) and 90% acetonitrile/0.1% formic acid (solvent B), and the 110-min LC-MS/MS method consisted of a 10-min column equilibration procedure, 10-min sample-loading procedure, and the following gradient profile: (min: B %) 0:0; 2:6; 72:40%; 78:90%; 84:90%; 87:50%; 90:50% (last three steps at 500 nL/min flow rate). The MS and MS/MS data were acquired in positive-ion mode at a spray voltage of 2.5 kV and at a resolution of 60,000 at m/z 400. For every duty cycle, the 10 most abundant peptide precursors were selected for MS/MS analysis in the LTQ Orbitrap Velos (normalized collision energy, 40%). A detailed flowchart of iTRAQ-based quantitative proteomics is shown (FIG. 4A).

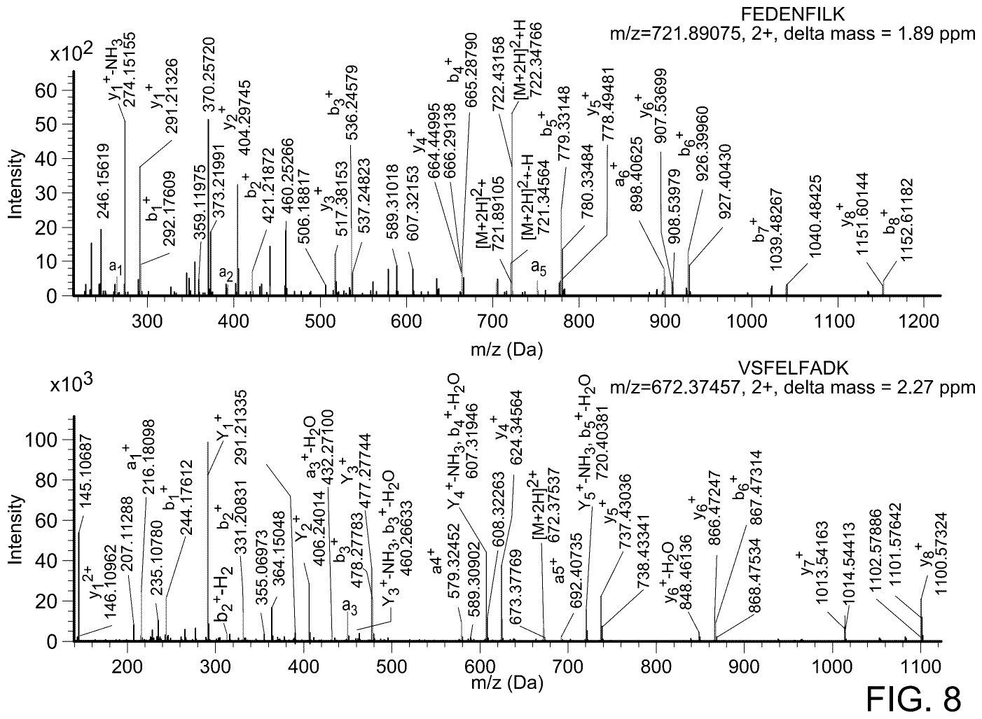

Quantitative Proteomics Analysis. The MS data from the iTRAQ experiments were analyzed with Proteome Discoverer (version 2.1; Thermo-Fisher). MS/MS spectral data were processed using the extract feature under the MASCOT and Sequest HT search components of the program. For both components, the same search parameters were selected, and these included iTRAQ labels at tyrosine, oxidations of methionine, and deamidation at N/Q as variable modifications. iTRAQ labels at N terminus, and lysine, methylthio label at cysteine were used as fixed modifications. The MS data were searched against NCBI RefSeq 72 human protein database containing 55,692 sequences. Proteome Discoverer calculates the percentage of false identifications using a separate decoy database (reverse database) that contains the reversed sequences of the protein entries. The Proteome Discoverer counts the number of matches from both searches and calculates the false-discovery rate (FDR) by counting only the top match per spectrum, assuming that only one peptide can be the correct match. The score thresholds were adjusted to obtain 1% and 5% reverse hits compared with forward hits, resulting in an overall FDR of 5%. Precursor and reporter ion window tolerance were fixed at 20 ppm and 0.05 Da, respectively. The criteria specified for generation of peak lists included signal-to-noise ratios of 1.5 and inclusions of precursor mass ranges of 600-8,000 Da. The two validated SAFE-SRM target peptides from PPIA protein were initially identified unambiguously using a 1% FDR cutoff, as shown in FIG. 8.

Selection of 641 Peptides as Potential Cancer Biomarkers for Further Validation. A total of 204 proteins was shared by at least two out of three whole-plasma iTRAQ proteomics datasets. Eighty-seven of these proteins were selected as potential cancer biomarkers for further SRM-based validation based on their abundance test score in the empirical modified eBayes t test. A total of 461 proteotypic peptides from these proteins was selected as SRM quantifying targets (approximately five target peptides per protein). Of these 461 peptides, 208 were directly observed in our experiments and an additional 253 peptides were added from querying several databases, including PeptideAtlas, PRIDE, etc. (see, e.g., Desiere et al. 2006 Nucleic Acids Res 34: D655-D658; Wang et al. 2011 Proc Natl Acad Sci USA 108:2444-2449; and Vizcaíno et al. 2016 Nucleic Acids Res 44: D447-D456). We also identified 180 peptides in our iTRAQ datasets that did not meet our rigorous criteria for initial selection but which we considered reasonable candidate biomarkers on the basis of their biologic properties. Altogether, we selected 641 SRM target peptides from phase 1 of our study that were carried forward to the validation phase (Dataset S4).

Statistical Analysis of Peptide Quantification Using the limma Package in R/Bioconductor. Peptide expression ratios of the pooled samples were calculated based on the median value of peptide ion intensities of iTRAQ labeling 117 (pancreatic cancer pool), 116 (colorectal cancer pool), or 115 (ovarian cancer pool) relative to that of 114 (normal individual pool). Sample preparation was performed in duplicate (two biological replicates). MS analysis was performed once on the first replicate (generating dataset 1) and twice on the second replicate, generating datasets 2 and 3, which were therefore technical replicates. A matrix was generated to store the raw peptide abundance data, where row names contained all unique sequences of the peptides. Columns 1 through 4 stored the intensities of 114, 115, 116, and 117 labeling intensities from dataset 1. Columns 5 through 8 and columns 9 through 12 stored the analogous labeling intensities from datasets 2 and 3, respectively. “NA” was used to indicate that a peptide was not detected in a particular dataset with a particular label (Dataset S2).

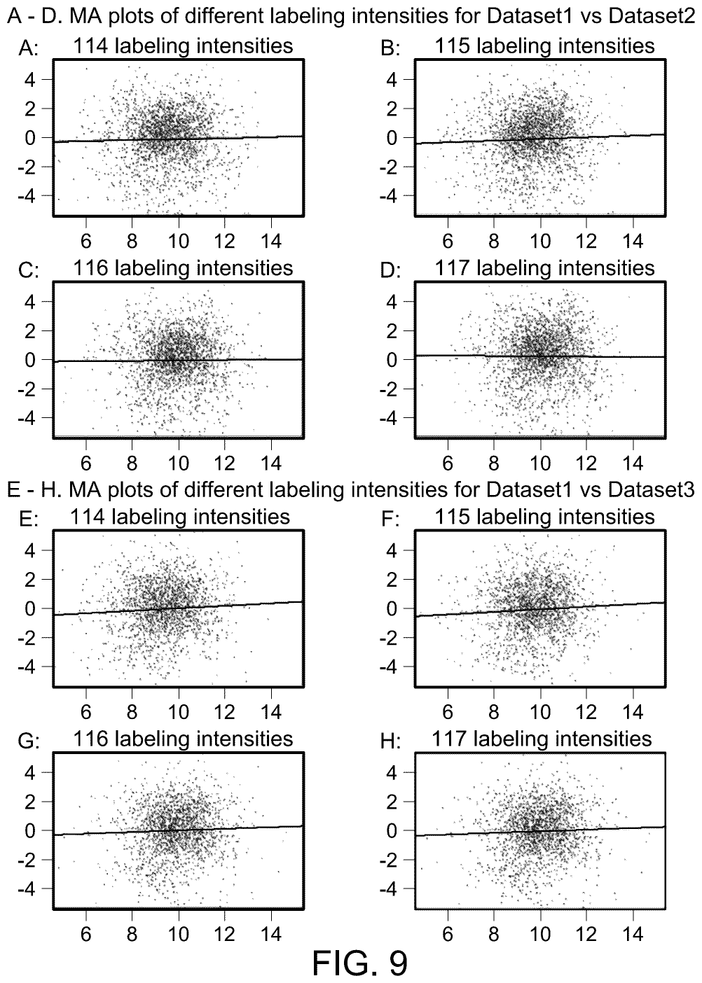

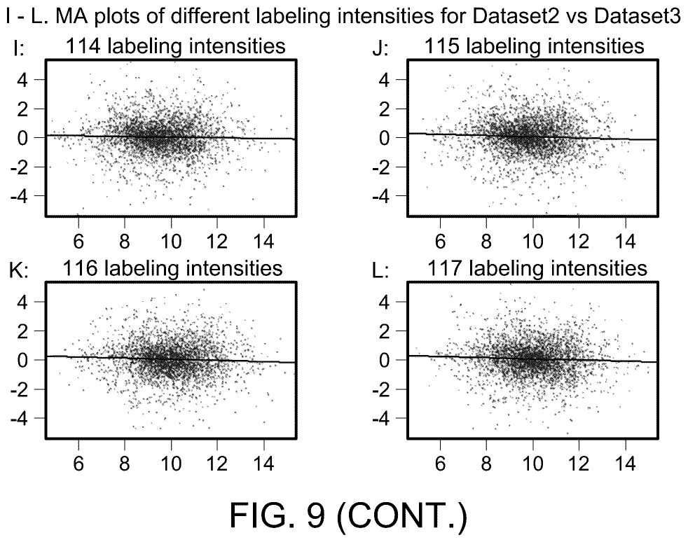

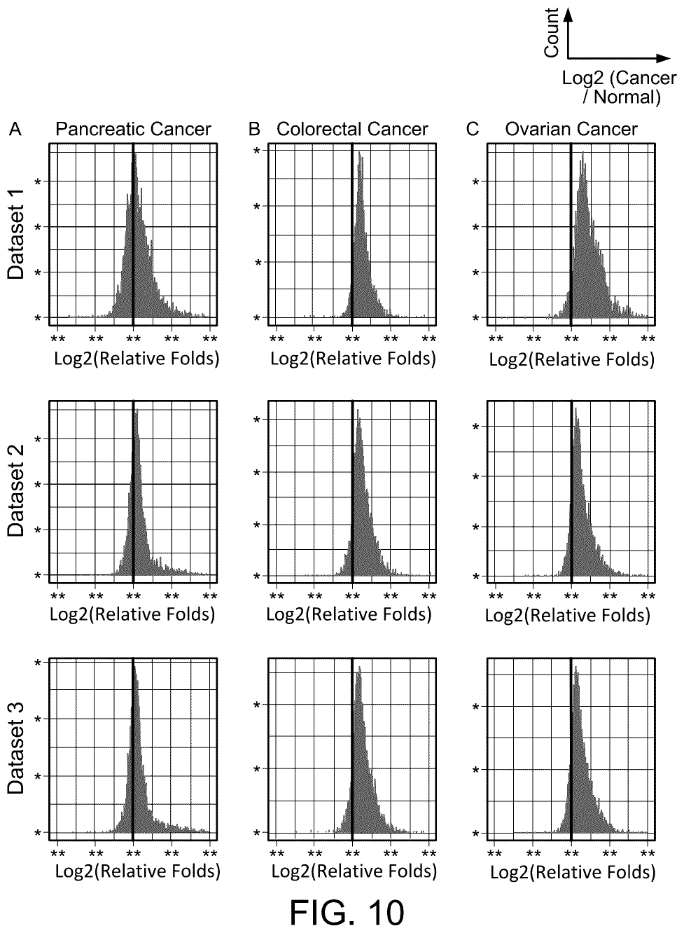

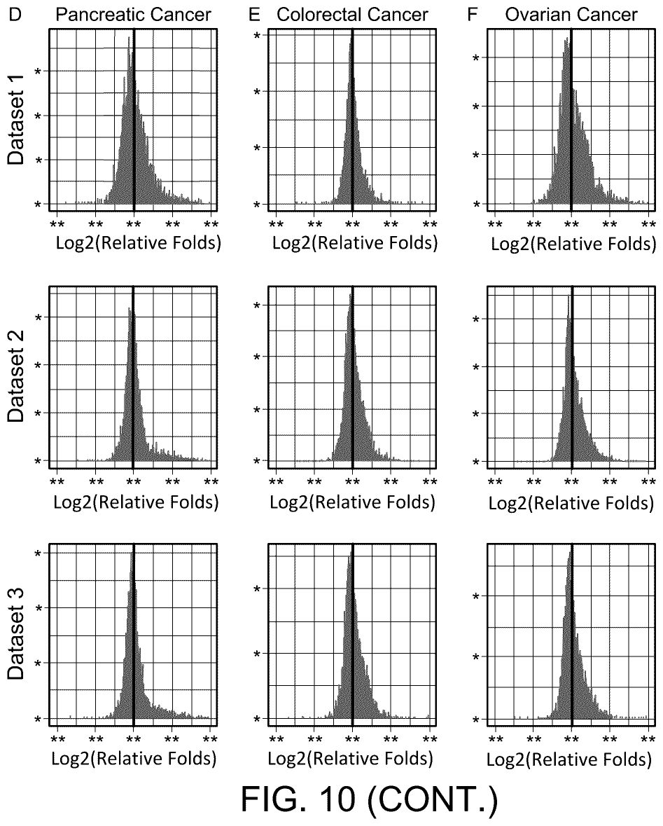

MA plots were generated to compare the potential bias between different datasets. Because no significant bias was observed in these MA plots (FIG. 9), median normalization was chosen for subsequent analysis (FIG. 10). For this analysis, we borrowed the concepts developed for the analysis of microarray data and used R packages from the Bioconductor project to analyze peptide fold changes (see, e.g., Li et al. 2009 Expert Rev Mol Diagn 9:555-566). In particular, we used the modified t test from limma (linear models for microarray data) to judge the statistical significance of the changes observed (see, e.g., Li et al. 2009 Expert Rev Mol Diagn 9:555-566).

Let yi and xi denote the abundances of the ith protein in cancer plasma proteome and normal plasma proteome, respectively, so that

$${y}_{j} \sim Norm ( {μ}_{yj} , \underline{{σ}_{vi}} )$$

$$and {x}_{i} \sim Norm ( {μ}_{xi} , \underline{{σ}_{xi}} )$$

where μ and σ denote the mean and variance of a peptide abundance in the three datasets. To avoid identifying peptide biomarkers (highly up-regulated in cancer plasma proteome compared with normal) that have significant variance between replicates, we adopted a t test where

$$t - statistic = \frac{\overline{y} - \overline{x}}{\sqrt{\frac{{{δ}_{x}}^{⌢} + {{δ}_{y}}^{⌢}}{n}}}$$

The t test was modified by an empirical Bayes method. Instead of testing each peptide in isolation from all others, the empirical Bayes modified t test borrows strength from all other peptides, thus improving the error estimate of each individual peptide. The eBayes modified t test from limma R package was used to perform statistical analysis for the difference of peptide abundances between samples. In total, 208 peptides from 87 different proteins were identified as candidate cancer biomarkers and were carried on to the validation phase of this study.

Candidate Biomarkers Identified by Quantitative Plasma Proteomics. Proteomics database searches (using PRIDE, www.ebi.ac.uk/pride/archive/, and Peptide Atlas, www.peptideatlas.org/) were conducted for the 87 proteins, and their 253 most readily detectable peptides (other than the 208 noted above) were added to the candidate peptide list. Another 180 peptides observed repeatedly from the three discovery datasets but that did not pass the eBayes modified t test were also added. In total, 641 candidate peptides were subject to further validation (Dataset S4).

Development of SAFE-SRM Assays. A total of 4,384 transitions targeting the 641 target peptides in our study was optimized by using synthetic peptides. For each synthetic peptide, a set of optimized collision energies and dwell times according was obtained (Dataset S5).

Briefly, an HPLC fractionation was performed to separate the 641 synthetic peptides into 96 fractions based on each peptide's hydrophobicity in a weak basic environment (pH 8.2). A total of 96 peptide fractions was then organized into 32 groups comprising three sequential fractions each, according to the scheme shown in FIG. 5. Each of these groups was subjected to fractionation through a C18-based HPLC coupled to the Agilent 6490 triple-quadrupole mass spectrometer. SRM assays covering all 4,384 transitions were performed in each of the groups to determine the optimum parameters for detecting each peptide. After identifying the SAFE-SRM fraction group ID for each peptide, a unique SAFE-SRM method was constructed for each fraction group, and the SRM transitions in sequential groups that eluted just before or just after the target group were also incorporated into the method (FIG. 5). The SAFE-SRM group ID for each peptide is listed in Dataset S5, where each ID refers to the bRPLC fractionation plate shown on FIG. 5.

The 641 candidate peptides were synthesized and used as standards to establish the SAFE-SRM method using a three-step optimization approach:

i) Optimization of collision energy was performed for each pair of precursor ion (usually positively charged proteotypic peptide) and product ion (peptide fragments generated from collision-induced dissociation). For each precursor ion, two steps above and two steps below (step size, 4 eV) the theoretical optimum value of collision energies were applied to fragment each precursor ion. For each peptide, five to eight fragmented ions showing the strongest intensities were selected as the detection targets. Mass-to-charge ratio (m/z) of the peptide, optimized collision energy values, and the m/z of the peptide fragmented ions were thus established for each peptide. A set of such values is typically termed as an SRM transition. In total, 4,384 SRM transitions were optimized in this way to target the 641 peptides (on average, approximately seven transitions per peptide).

ii) Optimization of bRPLC fractionation. The 641 synthetic peptides were spiked into the peptides derived from the pooled normal plasma sample used in phase 1 of the study prepared as described above, and three independent HPLC fractionations were carried out. As noted above, the 96 fractions from the bRPLC fractionation were combined into “fraction groups,” with each group containing three sequential fractions. The 4,384 transitions were assessed in each bRPLC fraction group, with fixed dwell time for each transition (5 ms). The bRPLC fraction group containing the highest amount of each peptide was determined, thereby defining a fraction group ID for each peptide. The standard intensity (SI) (the intensity measured by mass spectrometer for 10 fmol of the peptide) for each peptide was also recorded.

iii) SRM method assembly. A unique SRM method was created for each fraction group by compiling all of the transitions from the peptides with the same fraction group ID. The same SRM transitions were evaluated in the fraction groups eluting before and after the main fraction group. Thus, each fraction group was assessed with three different sets of SRM transitions. The dwell time for each transition was modified to be inversely proportional to the SI of the peptide, ranging from 3 to 20 ms.

For each synthetic peptide, a set of optimized collision energies and dwell times according was obtained. A list of the SRM transitions and fraction group IDs for all of the peptides are shown in Dataset S5. All transition parameters were manually examined and curated to exclude ions with excessive noise due to coelution with nonspecific analytes in human plasma samples. A set of 1,990 transitions was reproducibly detectable in a pool of all advanced cancer plasma samples used in phase 1, corresponding to 318 peptides (Dataset S5).

After initial method-building steps using standard peptides, we were able to pare the number of groups that needed to be analyzed in the final HPLC-MS step from 32 to 20. A total of 318 of the 641 peptides was reproducibly observed in at least one of these 20 groups, yielding 1,990 detectable transitions (average of 6.3 transitions per peptide).

Performance Evaluation of SAFE-SRM. Six heavy-isotope-labeled peptides (peptide 1: IQLVEEELDR* (SEQ ID NO:3); peptide 2: VILHLK* (SEQ ID NO:4); peptide 3: IILLFDAHK* (SEQ ID NO:5); peptide 4: TLAESALQLLYTAK* (SEQ ID NO:6); peptide 5: LLGHLVK* (SEQ ID NO:7); peptide 6: GLVGEIIK* (SEQ ID NO:8), where * indicates C13 and N15 heavy-isotope-labeled amino acids) were mixed at 1 fmol each, and the mixture was analyzed by a standard SRM method. Equal amounts (1 fmol each) of the six heavy-isotope-labeled peptides were spiked into proteolytically digested plasma peptide sample, followed by detection through a standard SRM approach (without bRPLC fractionation), a bRPLC-SRM approach, or a SAFE-SRM approach. The peptide abundance was calculated by the AUC of the peptide's SRM signal detected in each approach.

Agilent 6490 Mass Spectrometer Tuning. SAFE-SRM assays for each plasma sample were conducted only after confirmation of the instrument's performance with the manufacturer's tuning mixes (Autotune and Checktune) as well as a tuning mixture we prepared. Our tuning mixture was composed of 20 peptides representing a wide range of mass (M/z range, 200-1,400) and hydrophobicity (Table S2).

TABLE S2
Standard Peptides in Tuning Mixture (10 femto mole each).

| | SAFE-SRM | |
|---|---|---|
| Peptide Sequence | fraction group ID | SEQ ID NO: |
| DEIESVK | 3 | 9 |
| VGSAKPGLQK | 4 | 10 |
| ETIVLK | 5 | 11 |
| IQLVEEELDR | 6 | 3 |
| SIVNYKPK | 7 | 12 |
| DLQFVEVTDVK | 7 | 13 |
| TLLGDGPVVTDPK | 8 | 14 |
| GLVGEIIK | 9 | 8 |
| HFTILDAPGHK | 10 | 15 |
| LVDKFLEDVK | 11 | 16 |
| KIPVVFR | 11 | 17 |
| VILHLK | 12 | 4 |
| FPVIQHFK | 12 | 18 |
| LLGHLVK | 13 | 7 |
| FFLSHPAYR | 14 | 19 |
| LFAGLVHVK | 14 | 20 |
| TLAESALQLLYTAK | 15 | 6 |
| IILLFDAHK | 15 | 5 |
| VLDFEHFLPMLQTVAK | 17 | 21 |
| LLGNVLVCVLAHHFGK | 18 | 22 |

Data Analysis. A set of assays composed of 20 different SRM methods for all groups were performed to quantify the abundance of each of the 318 peptides. Twenty datasets were generated by the mass spectrometer using the 20 SAFE-SRM methods for each plasma sample and were imported into Skyline 3.6 for data analysis (see, e.g., MacLean et al. 2010 Bioinformatics 26:966-968). We improved the labeled reference peptide (LRP) method (see, e.g., Zhang et al. 2011 Mol Cell Proteomics 10: M110.006593) through a dual-control approach to adjust for the variance of sample preparation efficiency and fluctuations of mass spectrometer sensitivity. The first control was a heavy-isotope-labeled mutant KRAS protein spiked into the plasma sample before sample preparation. The second control was a heavy-isotope-labeled WT KRAS peptide spiked into each group before running on the final HPLC-MS. The abundance of a target peptide was represented by the total area under the curve (AUC) of all its transitions normalized to the total AUC of all transitions from the 3-fmol heavy-isotope (heavy-lysine residue)-labeled K-Ras WT peptides (LVVVGAGGVGK; SEQ ID NO:23). Variations in sample preparation were adjusted by normalizing the abundance of each peptide from a given sample to the abundance of the peptides derived from the heavyisotope-labeled K-Ras mutant (G12D) protein purchased from Origene. We selected six peptides derived from this heavy-isotope amino acid (heavy-lysine and heavy-arginine)-labeled protein for this adjustment. Peptide sequences and optimized transition parameters are listed in Dataset S5.

A SAFE-SRM abundance score(S) was calculated for each of the 318 peptides in every sample. Assume that Pi, j, k is the integrated intensity of a peptide i in sample j fraction k, Nj,k is the integrated intensity of the K-Ras WT heavy control peptide in sample j, fraction k, and Mj is the integrated intensity of the median abundance K-RAS protein peptide in sample j. Let Si,j be the abundance score of peptide i in sample j; therefore, Si,j can be calculated as follows:

$${S}_{i , j} = \frac{{P}_{i , j , k}}{{N}_{j , k} * {M}_{j}} ,$$

where for Mj:

$${M}_{j} = median ( \frac{{P}_{1 , j , k}}{{N}_{j , k}} , \frac{{P}_{2 , j , k}}{{N}_{j , k}} , … , \frac{{P}_{6 , j , k}}{{N}_{j , k}} ) .$$

In this study, 71 out of 318 peptides were repeatedly detected across two adjacent SAFE-SRM groups. The abundance of such peptides in each sample was calculated by summing the normalized abundance scores in adjacent SAFE-SRM runs where the peptides were detected.

Reproducibility of the SAFE-SRM pipeline was measured by calculating the reproducibility ratio (RR) for sample j as follows:

$$R {R}_{j} = median ( \frac{{N}_{j , k}}{{M}_{j}} ) .$$

RR value for each sample processed through SAFE-SRM pipeline was listed in Dataset S7.

Cancer Proteomic Biomarker Identification. To identify the best peptide classifiers, stepwise forward selection logistic regression was employed in MATLAB. First, a logistic regression model was fit to the training set of 50 samples, including 27 known healthy samples and 7, 7, and 9 known colorectal, ovarian, and pancreatic cancer plasma samples using the 318 peptide abundance scores. Leave-one-out cross-validation was used to estimate predictive performance of each model. The peptide yielding the lowest cross-validated misclassification rate on the training set was selected for inclusion in the model. If more than one peptide achieved the lowest misclassification rate, ties were broken by selecting the peptide that produced the greatest model likelihood. This process of selecting a peptide biomarker to be added to the model was repeated until no further decrease in cross-validated misclassification rate could be achieved by addition of a peptide. To find a subset of peptides from the same protein that could achieve perfect classification, the same stepwise forward selection procedure was applied for each potential biomarker protein. After identifying the best classifiers, predictive performance of models fit to different combinations of the peptide biomarkers was compared on an additional 48 samples in a blind manner. The predictive models constructed by combinations of best peptide classifiers and by each individual best peptide classifier were evaluated on an additional cohort of 73 samples in a blind manner.

### Results

Study Design. This study was designed to identify and validate unique proteomic biomarkers for cancers using a combination of qualitative and quantitative MS techniques. Most previous studies in this area have begun with the analysis of cancer tissues, and then attempted to determine whether cancer-specific proteins or peptides could be identified in the plasma. In the current study, we attempted to identify candidate peptides directly from the plasma. The study was executed in three discrete phases: phase 1, global plasma proteomic profiling of samples from cancer patients and healthy individuals, yielding 641 candidate peptide markers from 188 genes; phase 2, implementation of a selected reaction monitoring (SRM)-based assay, called sequential analysis of fractionated eluates by SRM (SAFE-SRM), to evaluate each of the 641 candidate peptide markers in additional plasma samples, yielding two peptides from peptidyl-prolyl cis-trans isomerase A (PPIA) as promising biomarkers; and phase 3, evaluation of the performance of these two peptides in an independent set of cancer patients and controls using SAFE-SRM. Phase 1 was performed on an Orbitrap mass spectrometer, which is most suitable for qualitative analysis of large numbers of proteins, while phases 2 and 3 were conducted on a triple-quadrupole mass spectrometer, most suitable for quantitative analyses of selected analytes. A total of 266 plasma samples from different donor sources was evaluated during the three phases of this study (Table S1).

TABLE S1
Study design and cases involved in the study.

| | | Normal | Pan- | Colo- | | |
|---|---|---|---|---|---|---|
| Study | | Healthy | creatic | rectal | Ovarian | |
| Phases | Study aims | Individual | Cancer | Cancer | Cancer | Total |
| Phase 1 | Identification | 50 | 13 | 18 | 18 | 99 |
| | of candidate | | | | | |
| | biomarkers | | | | | |
| | from cancer | | | | | |
| | patients | | | | | |
| Phase 2 | Development | 32 | 14 | 20 | 28 | 94 |
| | of SAFE-SRM | | | | | |
| | and Testing | | | | | |
| | of candidate | | | | | |
| | peptides by | | | | | |
| | SAFE-SRM | | | | | |
| Phase 3 | Validation by | 14 | 24 | 0 | 35 | 73 |
| | SAFE-SRM | | | | | |

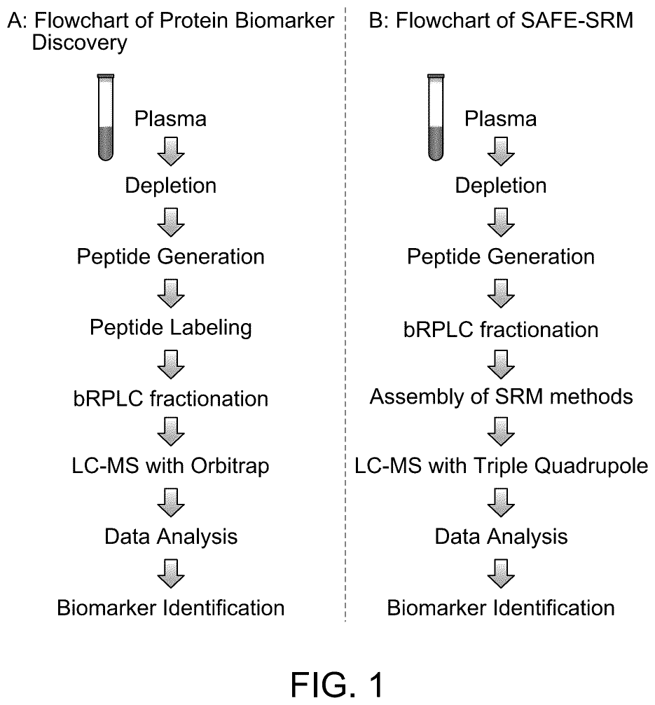

Phase 1: Identification of Candidate Biomarkers from Cancer Patients. To identify potential protein biomarkers for cancers, we first created four pooled human plasma samples composed of equal volumes of plasma from 50 normal healthy individuals, 18 patients with ovarian cancer, 13 patients with pancreatic cancer, and 18 patients with colorectal cancer (Dataset S1). All patients with cancer had advanced disease so as to maximize the likelihood that high concentrations of putative biomarkers would be found in the plasma. An antibody-based plasma depletion was performed to remove 14 highly abundant proteins, such as albumin and immunoglobulins, from each of the four pools. Each pool was then digested with trypsin and the resultant peptides were differentially labeled with iTRAQ. iTRAQ labeling allows the four pools to be mixed and analyzed in a single MS experiment. The pools were then analyzed to assess whole proteomes (FIG. 1A and FIG. 4A). In a separate experiment, the pooled plasma samples were enriched for glycoproteins before trypsin digestion and iTRAQ labeling to reveal potential differences in the peptides derived from glycosylated proteins (FIG. 4B).

We performed replicates of the entire workflow outlined in FIG. 4. In total, 223,602 peptides were identified through these analyses, representing 10,789 unique peptides from 1,249 unique proteins (Datasets S2 and S3). The relative abundances of each of these peptides in the plasma samples from cancer patients and normal individuals was then calculated using an empirical-Bayes modified t test (Materials and Methods). A total of 8,069 unique peptides was quantified in at least two replicates, and the correlation for the abundances of these peptides between the replicates was 0.74 (95% CI, 0.73-0.75). As described in detail in Materials and Methods, our analyses eventually yielded 641 peptides derived from 188 proteins with significantly increased abundance in the pooled cancer plasma samples compared with the pooled normal controls (Dataset S4).

Phase 2a: Development of SAFE-SRM. The validation of hundreds of unique potential peptide biomarkers is a daunting task. This difficulty is exacerbated by the fact that the abundances of such peptides from plasma proteins are generally low and the abundances of different peptides vary considerably within this low range. We developed an approach to tackle these challenges, with five major components. First, the 641 peptides of interest were individually synthesized, but not highly purified, so as to keep costs manageable. Second, an SRM method was created for each of these peptides. Each of the 641 methods was optimized for the collision energies and dwell times of the precursor ions that yielded the highest intensities of the post-collision peptide-specific transitions of major interest. The dwell time given to each peptide was inversely proportional to the peptide's intensity measured from a human plasma peptide sample spiked with equal amounts of synthetic peptides. This feature permitted the instrument to spend more time on detecting the peptides with lower signal intensities, thereby improving the overall ion statistics for the detection of low-abundance peptides. This protocol led to the identification of 4,384 transitions (approximately seven transitions per peptide; Dataset S5).

Third, the peptides were fractionated using basic pH reversed-phase liquid chromatography (bRPLC), yielding 96 fractions organized into 32 “fraction groups” each containing three sequential fractions; 20 fraction groups were selected for further analysis. Fourth, the peptides in each fraction group were separated by an orthogonal high-performance liquid chromatography (HPLC) method based on hydrophobic interactions (C18-RPLC). Finally, continuous elutes from the second HPLC column were analyzed using an SRM method composed of the collision energies, dwell times, and transitions that had been preoptimized using the synthetic peptides noted above. We termed this approach SAFE-SRM (FIG. 5). One advantage of SAFE-SRM is that it employs a two-dimensional chromatographic fractionation. The individual fractions contain much less peptide than the total, thereby reducing ion suppression from unwanted peptides and increasing the signal-to-noise ratio. A second advantage of SAFE-SRM is that it converts the qualitative approach used for peptide discovery to a quantitative approach during the validation phases. Finally, the method is highly tolerant to fluctuations in elution times that are commonly observed in bRPLC chromatography because sequential fractions are redundantly tested for peptide abundance (Materials and Methods).

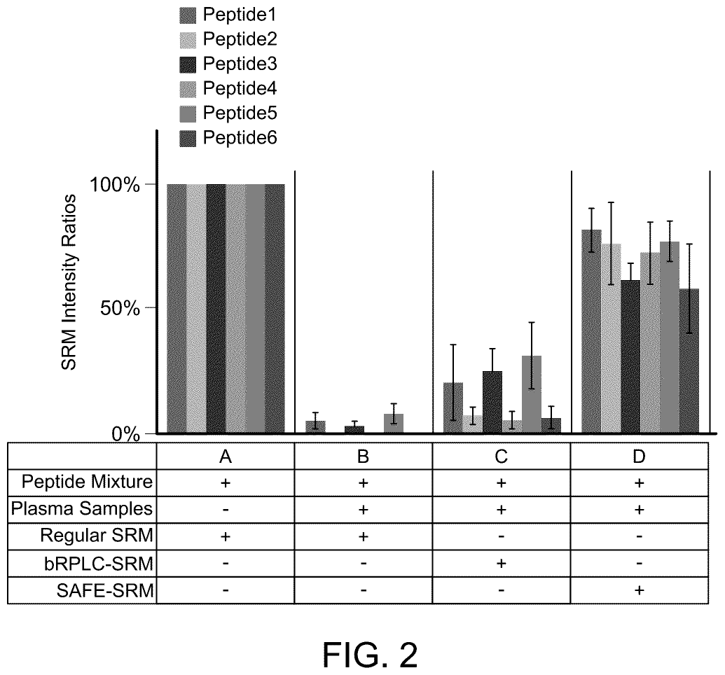

To assess the performance of SAFE-SRM, we chose six peptides with different hydrophobicity characteristics in HPLC and synthesized them as heavy isotope-labeled forms (Materials and Methods). We then mixed these peptides and performed a standard SRM analysis using the optimized collision energies and dwell times described above. All six peptides were detected at high confidence, as expected. However, when we spiked these peptides into trypsin-digested samples generated from normal plasma as described above, their average intensities were only around 5% of that obtained with the pure peptides, and only three of the six peptides were detectable at all. When this spiked sample was analyzed with SAFE-SRM, all six peptides could be detected, with an intensity that averaged 70% of that obtained with the pure peptides (FIG. 2).

Phase 2b: Testing of Candidate Peptides by SAFE-SRM. We began by using SAFE-SRM to evaluate the four plasma pools used for the initial iTRAQ-based discovery phase of the study. We expected that the peptides detectable in these pooled samples would be those least likely to be affected by ion suppression, the coelution of unwanted peptides in the same chromatographic fractions, or other technical issues. After careful examination, 318 out of the 641 tested peptides proved to be reproducibly detectable in the pooled samples through 1,990 transitions (6.3 transitions per peptide; Dataset S5). These 318 peptides mapped to 121 proteins.

We then used SAFE-SRM to evaluate 94 individual plasma samples, none of which was used in the discovery phase. Forty eight of these samples were from normal individuals and 14, 14, and 18 were from patients with colorectal cancers, ovarian cancers, and pancreatic cancers, respectively (Dataset S1). SAFE-SRM abundance scores were calculated for each of the 318 peptides in each of the 94 individual and 4 pooled plasma samples (Dataset S6). We used statistical methods to determine whether any peptide or combination of peptides was able to accurately classify the origin of a sample from the peptide signatures. For this purpose, we randomly selected approximately one-half of the samples for training (27 from healthy donors and 7, 7, and 9 samples from patients with colorectal cancers, ovarian cancers, or pancreatic cancers, respectively). The remaining half of the samples were used to test the performance of the classifiers derived from the training samples.

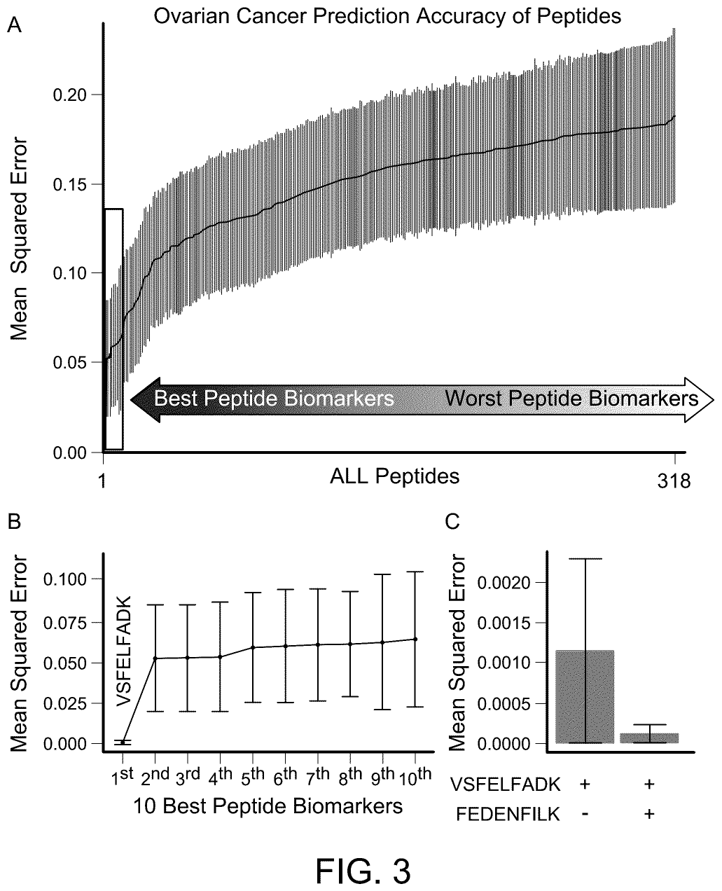

A recursive, leave-one-out cross-validation strategy was used to estimate the predictive performance of the classification model as it evolved. The peptides yielding the highest cross-validated classification scores on the training set were first selected. Data on the peptides were then searched to determine whether any second peptide could increase the classification score. This process of selecting a peptide biomarker to be added was repeated until no further increases in the classification score could be achieved by addition of other peptides. Using this approach, several combinations of peptides with excellent classification potential were identified (FIGS. 3A and B).

The best performance was observed for the classification of ovarian cancers with a combination of several markers. The top single peptide marker for ovarian cancers was VSFELFADK (SEQ ID NO:1) from PPIA (also known as Cyclophilin-A). We then determined whether any of the other peptides from PPIA among those in the 318-peptide set could be added to the classifier without decreasing specificity and found that a second peptide from PPIA (FEDENFILK; SEQ ID NO:2) could be added in this way (FIG. 3C). Using peptide abundance levels resulting in 100% specificity among 36 normal samples, we found that VSFELFADK (SEQ ID NO:1) and FEDENFILK (SEQ ID NO:2) yielded 75.0% and 78.6% sensitivities, respectively. The Pearson correlation coefficient for the two PPIA peptides was 0.83 (95% CI, 0.78-0.87). At least one of the two peptides was elevated in 23 (82.1%) of the 28 samples.

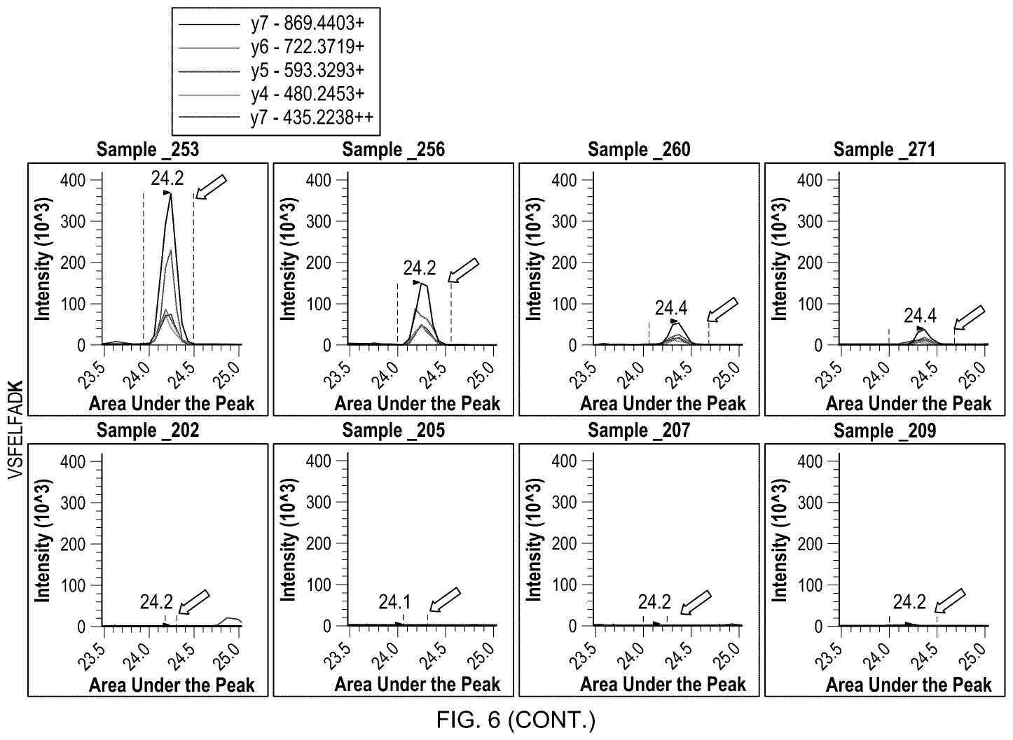

Phase 3: Validation. The dataset used to form the classifier was large: 1,990 transitions from 318 peptides tested in each of 98 samples. It is well known that overfitting is possible in such experiments and that independent validations of any classifier are mandatory. We therefore evaluated a separate cohort of 73 cases, consisting of plasma from 35 ovarian cancer cases and 38 samples from healthy individuals or patients with other cancer types (Dataset S7). In these 73 cases, SAFE-SRM was performed, but the only transitions analyzed were those corresponding to the two peptides from PPIA plus a peptide from Fibronectin, which we found to be expressed at similar levels in all samples and was thereby used for normalization. The relative abundances required for a positive score were predetermined from the results in phase 2b described above. Examples of the SAFE-SRM profiles for these peptides in ovarian cancer patients and normal individuals are shown in FIG. 6. Twenty (57.1%; 95% CI, 40-73%) of the 35 plasma samples from ovarian cancer cases scored positive for VSFELFADK (SEQ ID NO:1) from PPIA, while none of the 14 samples from normal individuals scored positive (specificity of 100%; 95% CI, 89-100%). For the second peptide FEDENFILK (SEQ ID NO:2) from PPIA, 14 (40.0%; 95% CI, 24-58%) of the 35 plasma samples from ovarian cancer cases were scored as positive, and, as for the first PPIA peptide, none of the 14 samples from healthy individuals scored positive. All of the plasma samples scoring positive for the FEDENFILK (SEQ ID NO:2) peptide also scored positive for the VSFELFADK (SEQ ID NO:1) from the same protein. Twenty-four patients with pancreatic cancer were tested in this assay, and only one of them (4.2%; 95% CI, 0.2-23.1%) scored positive for peptide VSFELFADK (SEQ ID NO:1), and none for peptide FEDENFILK (SEQ ID NO:2) (Dataset S7).

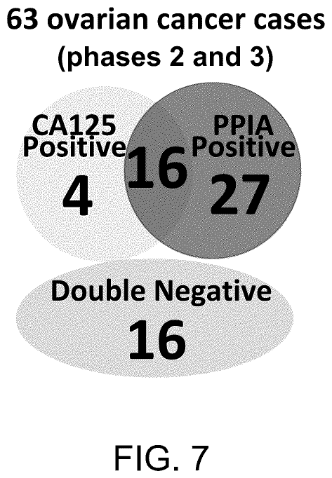

It was notable that 11 of 17 (64.7%) of the plasmas from patients with early-stage ovarian cancers scored positive for PPIA peptides, while 32 of 46 (69.6%) of the plasmas from patients with more advanced cancers scored positive (combining phase 2b and phase 3; Dataset S7). For comparison, CA125 levels were measured in a subset of the same cohort. CA125 was elevated in 20 of 63 ovarian cancer patients and in none of 50 healthy controls. The elevations in CA125 and PPIA did not completely overlap, so that the sensitivity for detection of either CA125 or PPIA levels was 74.6% (95% CI, 62.1-84.7%), higher than either alone (see Venn diagram in FIG. 7).

These results demonstrate that SAFE-SRM can be used as a generalizable method for discovering disease-specific peptides in the circulation. Specifically, the SAFE-SRM method was used to identify and validate peptides from PPIA that can be used as a circulating peptide marker to identify mammals as having ovarian cancer.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating ovarian cancer, said method comprising:
detecting an elevated level of one or more peptide biomarkers comprising a peptide fragment derived from a peptidyl-prolyl cis-trans isomerase A (PPIA) polypeptide in a blood sample obtained from a mammal; and
administering one or more cancer treatments to said mammal.

2. The method of claim 1, wherein said one or more cancer treatments are selected from the group consisting of: surgery, chemotherapy, hormone therapy, targeted therapy, radiation therapy, and combinations thereof.

3. (canceled)

4. The method of claim 1, wherein said mammal is a human.

5. The method of claim 1, wherein said blood sample is a plasma sample.

6. The method of claim 1, wherein said PPIA peptide fragment comprises amino acid sequence VSFELFADK (SEQ ID NO: 1).

7. The method of claim 1, wherein said PPIA peptide fragment comprises amino acid sequence FEDENFILK (SEQ ID NO: 2).

8. A method for identifying a peptide biomarker, said method comprising:
digesting polypeptides present in a disease blood sample to obtain disease peptide fragments;
labeling said disease peptide fragments with a first heavy isotope to obtain labeled disease peptide fragments;
digesting polypeptides present in a reference blood sample to obtain reference peptide fragments;
labeling said reference peptide fragments with a second heavy isotope to obtain labeled reference peptide fragments;
subjecting the labeled disease peptide fragments and the labeled reference peptide fragments to mass spectrometry to identify a peptide biomarker, wherein the level of said peptide biomarker is elevated in the labeled disease peptide fragments relative to the labeled reference peptide fragments.

9. The method of claim 8, wherein said disease blood sample comprises blood from one or more mammals having said disease.

10. The method of claim 9, wherein said disease blood samples comprises blood from a plurality of mammals having said disease.

11. The method of claim 8, wherein said reference blood sample comprises blood from one or more healthy mammals.

12. The method of claim 11, wherein said reference blood sample comprises blood from a plurality of healthy mammals.

13. The method of claim 8, wherein said method further comprises depleting one or more highly abundant blood proteins from each sample.

14. The method of claim 13, wherein said highly abundant blood proteins are selected from the group consisting of: albumin, IgG, α1-antitrypsin, IgA, IgM, transferrin, haptoglobin, α2-macroglobulin, fibrinogen, complement C3, α1-acid glycoprotein, apolipoprotein A-I, apolipoprotein A-II, apolipoprotein B, and combinations thereof.

15. The method of claim 8, wherein said method further comprises, prior to each digestion step, enriching glycoproteins in each sample.

16. The method of claim 8, wherein said mass spectrometry is performed using an Orbitrap mass spectrometer.

17. (canceled)

18. A method for identifying and validating a peptide biomarker, said method comprising:
A. identifying a candidate peptide biomarker, wherein said identifying comprises:
i. digesting polypeptides present in a disease blood sample to obtain disease peptide fragments;
ii. labeling said disease peptide fragments with a first heavy isotope to obtain labeled disease peptide fragments;
iii. digesting polypeptides present in a reference blood sample to obtain reference peptide fragments;
iv. labeling said reference peptide fragments with a second heavy isotope to obtain labeled reference peptide fragments;
v. subjecting the labeled disease peptide fragments and the labeled reference peptide fragments to mass spectrometry to identify a candidate peptide biomarker, wherein the level of said candidate peptide biomarker is elevated in the labeled disease peptide fragments relative to the labeled reference peptide fragments;
B. building a SAFE-SRM method, wherein said building comprises:
i. synthesizing said candidate peptide biomarker;
ii. subjecting said synthetic candidate peptide biomarker to mass spectrometry to determine a candidate peptide biomarker transition, wherein said transition is determined by identifying a precursor-product ion pair having a strongest intensity and identifying a collision energy (CE) producing said precursor-product ion pair;
iii. subjecting a plurality of peptides comprising said candidate peptide biomarker to basic pH reversed-phase liquid chromatography (bRPLC) to obtain a plurality of fractions, wherein said plurality consists of essentially equal amounts of each peptide;
iv. organizing said plurality of fractions into a plurality of fraction groups, wherein the number of fractions is higher than the number of fraction groups;
v. determining an intensity of said candidate peptide biomarker in each of said fraction groups using the candidate peptide biomarker transition and a fixed dwell time; and
vi. optimizing the dwell time by re-assembling the transitions according to their hydrophobicity at high pH; and
C. validating said candidate peptide biomarker, wherein said validating comprises:
i. quantitating said candidate peptide biomarker in said disease blood sample, said quantitating comprising:
a. subjecting said disease peptide fragments comprising said candidate peptide biomarkers to bRPLC to obtain a plurality of fractions;
b. organizing said plurality of fractions into a plurality of fraction groups, wherein the number of fractions is higher than the number of fraction groups;
c. separating peptides in each fraction group by orthogonal HPLC at acidic pH to obtain continuous HPLC elutes; and
d. analyzing said continuous HPLC elutes using a SRM method comprising said candidate peptide biomarker transition and said optimized dwell time;
ii. quantitating said candidate peptide marker in said reference blood sample, said quantitating comprising:
a. subjecting said reference peptide fragments to bRPLC to obtain a plurality of fractions;
b. organizing said plurality of fractions into a plurality of fraction groups, wherein the number of fractions is higher than the number of fraction groups;
c. separating peptides in each fraction group by orthogonal HPLC at acidic pH to obtain continuous HPLC elutes;
d. analyzing said continuous HPLC elutes using said SRM method comprising said candidate peptide biomarker transition and said optimized dwell time; and
iii. validating said candidate peptide biomarker when the candidate peptide biomarker is quantitated at an elevated level in said disease sample relative to said reference sample.

19. The method of claim 18, wherein said synthesized candidate peptide biomarkers are not labeled with a heavy isotope.

20-21. (canceled)

22. The method of claim 19, wherein said HPLC is performed with a device, which device is coupled to a mass spectrometer.

23. (canceled)

24. The method of claim 19, wherein the collision energy is any one of the collision energies set forth in Dataset S5.

25. The method of claim 19, wherein the dwell time is any one of the dwell times set forth in Dataset S5.
